SEQUENCE LISTING (15 sequences)

SEQ ID NO: 1 - protein, 476 aa, Homo sapiens
MMSFGGADALLGAPFAPLHGGGSLHYALARKGGAGGTRSAAGSSSGFHSWTRTSVSSVSASPSRFRGAGAASSTDSLDTLSNGPEGCMVAVATSRSEKEQLQALNDRFAGYIDKVRQLEAHNRSLEGEAAALRQQQAGRSAMGELYEREVREMRGAVLRLGAARGQLRLEQEHLLEDIAHVRQRLDDEARQREEAEAAARALARFAQEAEAARVDLQKKAQALQEECGYLRRHHQEEVGELLGQIQGSGAAQAQMQAETRDALKCDVTSALREIRAQLEGHAVQSTLQSEEWFRVRLDRLSEAAKVNTDAMRSAQEEITEYRRQLQARTTELEALKSTKDSLERQRSELEDRHQADIASYQEAIQQLDAELRNTKWEMAAQLREYQDLLNVKMALDIEIAAYRKLLEGEECRIGFGPIPFSLPEGLPKIPSVSTHIKVKSEEKIKVVEKSEKETVIVEEQTEETQVTEEVTEEEEK

SEQ ID NO: 2 - protein, 359 aa, Homo sapiens
EAKEEEGKEEEGGEEEEAEGGEEETKSPPAEEAASPEKEAKSPVKEEAKSPAEAKSPEKEEAKSPAEVKSPEKAKSPAKEEAKSPPEAKSPEKEEAKSPAEVKSPEKAKSPAKEEAKSPAEAKSPEKAKSPVKEEAKSPAEAKSPVKEEAKSPAEVKSPEKAKSPTKEEAKSPEKAKSPEKAKSPEKEEAKSPEKAKSPVKAEAKSPEKAKSPVKAEAKSPEKAKSPVKEEAKSPEKAKSPVKEEAKSPEKAKSPVKEEAKTPEKAKSPVKEEAKSPEKAKSPEKAKTLDVKSPEAKTPAKEEARSPADKFPEKAKSPVKEEVKSPEKAKSPLKEDAKAPEKEIPKKEEVKSPVKEEEK

SEQ ID NO: 3 - protein, 17 aa, Homo sapiens
PQEVKVKEPPKKAEEEK

SEQ ID NO: 4 - protein, 134 aa, Homo sapiens
APATPKTEEKKDSKKEEAPKKEAPKPKVEEKKEPAVEKPKESKVEAKKEEAEDKKKVPTPEKEAPAKVEVKEDAKPKEKTEVAKKEPDDAKAKEPSKPAEKKEAAPEKKDTKEEKAKKPEEKPKTEAKAKEDDK

SEQ ID NO: 5 - protein, 40 aa, Homo sapiens
TLSKEPSKPKAEKAEKSSSTDQKDSKPPEKATEDKAAKGK

SEQ ID NO: 6 - protein, 1026 aa, Homo sapiens
MMSFGGADALLGAPFAPLHGGGSLHYALARKGGAGGTRSAAGSSSGFHSWTRTSVSSVSASPSRFRGAGAASSTDSLDTLSNGPEGCMVAVATSRSEKEQLQALNDRFAGYIDKVRQLEAHNRSLEGEAAALRQQQAGRSAMGELYEREVREMRGAVLRLGAARGQLRLEQEHLLEDIAHVRQRLDDEARQREEAEAAARALARFAQEAEAARVDLQKKAQALQEECGYLRRHHQEEVGELLGQIQGSGAAQAQMQAETRDALKCDVTSALREIRAQLEGHAVQSTLQSEEWFRVRLDRLSEAAKVNTDAMRSAQEEITEYRRQLQARTTELEALKSTKDSLERQRSELEDRHQADIASYQEAIQQLDAELRNTKWEMAAQLREYQDLLNVKMALDIEIAAYRKLLEGEECRIGFGPIPFSLPEGLPKIPSVSTHIKVKSEEKIKVVEKSEKETVIVEEQTEETQVTEEVTEEEEKEAKEEEGKEEEGGEEEEAEGGEEETKSPPAEEAASPEKEAKSPVKEEAKSPAEAKSPEKEEAKSPAEVKSPEKAKSPAKEEAKSPPEAKSPEKEEAKSPAEVKSPEKAKSPAKEEAKSPAEAKSPEKAKSPVKEEAKSPAEAKSPVKEEAKSPAEVKSPEKAKSPTKEEAKSPEKAKSPEKAKSPEKEEAKSPEKAKSPVKAEAKSPEKAKSPVKAEAKSPEKAKSPVKEEAKSPEKAKSPVKEEAKSPEKAKSPVKEEAKTPEKAKSPVKEEAKSPEKAKSPEKAKTLDVKSPEAKTPAKEEARSPADKFPEKAKSPVKEEVKSPEKAKSPLKEDAKAPEKEIPKKEEVKSPVKEEEKPQEVKVKEPPKKAEEEKAPATPKTEEKKDSKKEEAPKKEAPKPKVEEKKEPAVEKPKESKVEAKKEEAEDKKKVPTPEKEAPAKVEVKEDAKPKEKTEVAKKEPDDAKAKEPSKPAEKKEAAPEKKDTKEEKAKKPEEKPKTEAKAKEDDKTLSKEPSKPKAEKAEKSSSTDQKDSKPPEKATEDKAAKGK

SEQ ID NO: 7 - protein, 1019 aa, Homo sapiens
MGSKRGISSRHHSLSSYEIMFAALFAILVVLCAGLIAVSCLTIKESQRGAALGQSHEARATFKITSGVTYNPNLQDKLSVDFKVLAFDLQQMIDEIFLSSNLKNEYKNSRVLQFENGSIIVVFDLFFAQWVSDENVKEELIQGLEANKSSQLVTFHIDLNSVDILDKLTTTSHLATPGNVSIECLPGSSPCTDALTCIKADLFCDGEVNCPDGSDEDNKMCATVCDGRFLLTGSSGSFQATHYPKPSETSVVCQWIIRVNQGLSIKLSFDDFNTYYTDILDIYEGVGSSKILRASIWETNPGTIRIFSNQVTATFLIESDESDYVGFNATYTAFNSSELNNYEKINCNFEDGFCFWVQDLNDDNEWERIQGSTFSPFTGPNFDHTFGNASGFYISTPTGPGGRQERVGLLSLPLDPTLEPACLSFWYHMYGENVHKLSINISNDQNMEKTVFQKEGNYGDNWNYGQVTLNETVKFKVAFNAFKNKILSDIALDDISLTYGICNGSLYPEPTLVPTPPPELPTDCGGPFELWEPNTTFSSTNFPNSYPNLAFCVWILNAQKGKNIQLHFQEFDLENINDVVEIRDGEEADSLLLAVYTGPGPVKDVFSTTNRMTVLLITNDVLARGGFKANFTTGYHLGIPEPCKADHFQCKNGECVPLVNLCDGHLHCEDGSDEADCVRFFNGTTNNNGLVRFRIQSIWHTACAENWTTQISNDVCQLLGLGSGNSSKPIFSTDGGPFVKLNTAPDGHLILTPSQQCLQDSLIRLQCNHKSCGKKLAAQDITPKIVGGSNAKEGAWPWVVGLYYGGRLLCGASLVSSDWLVSAAHCVYGRNLEPSKWTAILGLHMKSNLTSPQTVPRLIDEIVINPHYNRRRKDNDIAMMHLEFKVNYTDYIQPICLPEENQVFPPGRNCSIAGWGTVVYQGTTANILQEADVPLLSNERCQQQMPEYNITENMICAGYEEGGIDSCQGDSGGPLMCQENNRWFLAGVTSFGYKCALPNRPGVYARVSRFTEWIQSFLH

SEQ ID NO: 8 - protein, 1135 aa, Canis familiaris
MMSFSGADALLGAPFAPLHGGGSLHYALARKGGTRSTAGSSSGFHSWARTSVSSVSASPSRFRGAAATSSTDSLDTLSNGPEGCVVAAAAARSEKEQLQALNDRFAGYIDKVRQLEAHNRSLEGEAAALRQQHAGRAAMGELYEREVREMRGAVLRLGAARGQLRLEQEHLLEDIAHVRQRLDDEARQREEAEAAARALARFAQEAEAARVELQKKAQALQEECGYLRRHHQEEVGELLGQIQGCGAAQAQAQAEARDALKCDVTSALREIRAQLEGHAVQSTLQSEEWFRVRLDRLSEAAKVNTDAMRSAQEEITEYRRQLQARTTELEALKGTKDSLERQRSELEDRHQADIASYQEAIQQLDTELRNTKWEMAAQLREYQDLLNVKMALDIEIAAYRKLLEGEECRIGFGPSPFSLPEGLPKIPSTSTHIKVKSEEKIKVVEKSEKETVILEEQTEEIQVTEEVTEEEEKEAKEEKGEEEEAEEGEEETKSPPAEEAASPEKEEAKSPEKAKSPMKEEAKSPAEAKSPVKEEAKSPAEVKSPEKAKSPMKEEAKSPTEVKSPEKAKSPAKEEAKSPVEAKSPEKAKSPVKEEAKSPEKAKSPVKEEAKSPEKAKSPVKEEAKSPEKAKSPEKAKSPVKEEAKSPEKAKSPVKEEAKSPEKAKSPVKEEAKSPEKTKSPVKEEAKSPEKAKSPEKAKSPVKEEAKSPEKAKSPVKEEAKSPEKAKSPVKEEAKSPEKAKSPVKEEAKSPEKAKSPVKEEAKSPEKPKSPVKEEAKSPEKAKSPEKAKSPVKEEAKSPEKAKSPVKEEAKSPEKAKSPVKEEAKSPEKAKSPVKEEAKSPEKAKSPVKEEAKSPEKAKSPEKAKSPVKEEAKSPEKAKSPEKAKSPVKEEAKSPEKAKSPEKVKSPVKEETKAPEKEVTKKEEAKSPIKEEEKPQEVKVKEPAKKAEEEKAPATPKTEEKKDSKKDEVPKKEAPKPEVPEKKEPAVEKPKESKVEAKKETEDKKKAVTPEKEVPAKVKEEAKPKEKAEVAKKEQDDAKAKEPSKAAEKEPEKPKKEGTPAAPEKKDVKEEKTPEAKKSEEKPKAEAPAKEEPSKEAPTPGKAKTEKAEKSSSTDQKDSRPAEKATEDKASKGEK

SEQ ID NO: 9 - protein, 1018 aa, Pan troglodytes
  misc_feature (224)..(224): Xaa can be any naturally occurring amino acid
MMSFGGADALLGAPFAPLHGGGSLHYALARKGGAGGTRSAAGSSSGFHSWTRTSVSSVSASPSRFRGAGAASSTDSLDTLSNGPEGCMVAVATSRSEKEQLQALNDRFAGYIDKVRQLEAHNRSLEGEAAALRQQQAGRSAMGELYEREVREMRGAVLRLGAARGQLRLEQEHLLEDIAHVRQRLDDEARQREEAEAAARALARFAQEAEAARVDLQKKAQALXEECGYLRRHHQEEVGELLGQIQGSGXXXXXXXXXXRDALKCDVTSALREIRAQLEGHAVQSTLQSEEWFRVRLDRLSEAAKVNTDAMRSAQEEITEYRRQLQARTTELEALKSTKDSLERQRSELEDRHQADIASYQEAIQQLDAELRNTKWEMAAQLREYQDLLNVKMALDIEIAAYRKLLEGEECRIGFGPIPFSLPEGLPKIPSVSTHIKVKSEEKIKVVEKSEKETVIVEEQTEETQVTEEVTEEEEKEAKEEEGKEEEGGEEEEAEGGEEETKSPPAEEAASPEKEAKSPVKEEAKSPAEAKSPEKEEAKSPAEVKSPEKAKSPAKEEAKSPPEAKSPEKEEAKSPVEVKSPEKAKSPAKEEAKSPAEAKSPEKAKSPVKEEAKSPAEAKSPVKEEAKSPAEVKSPEKAKSPTKEEAKSPEKSPEKAKSPVKAEAKSPEKAKSPVKAEAKSPEKAKSPVKAEAKSPEKAKSPVKAEAKSPEKAKSPVKEEAKTPEKAKSPVKEEAKSPEKAKSPVKEEAKSPEKAKTLDVKSPEAKTPAKEEARSPADKSPEKAKSPVKEEVKSPEKAKSPLKEDAKAPEKEIPKKEEVKSPVKEEEKPQEVKVKEPPKKAEEEKAPATPKTEEKKDSKKEEAPKKEAPKPKVEEKKEPAVEKPKESKVEAKKEEAEDKKKVPTPEKEAPAKVEVKEDAKPKEKTEVAKKEPDDAKAKEPSRPAEKKEAAPEKKDTKEEKAKKPEEKPKTEAKAKEDDKTLSKEPSKPKAEKAEKSSSTDQKDSKPPEKATEDKAAKGK

SEQ ID NO: 10 - protein, 1023 aa, Bos taurus
MSFSGADALLGAFAPLHGGGSLHYALARKGGAGGARSAAGSSSGFHSWARTSVSSVSASPSRFRGAGTTSSTDSLDTLSNGPEGCVVVARSEKEQLQVLNDRFAGYIDKVRQLEAHNRSLEGEAAALRQQQAGRAAMGELYEREVREMRGAVLRLGAARGQLRLEQEHLLEDIAHVRQRLDDEARQREEAEAATRALARFAQEAEAARVELQKKAQALQEECGYLRRHHQEEVGELLGQIQSSSAAQTQAEARDALKCDVTSALREIRAQLEGHTVQSTLQSEEWFRVRLDRLSEAAKVNTDAMRSAQEEISEYRRQLQARTTELETLKSTKDSLERQRSELEDRHQADIASYQEAIQQLDAELRNTKWEMAAQLREYQDLLNVKMALDIEIAAYRKLLEGEECRIGFGPSPFLLPEGLPKIPSVSTHIKVKSEEKIKMVEKSEKETVIVEEQTEEVQVTEEVTEEEEKEAKEEEGEAVKSPPAEEAASPEKEEAKSPAEAKSPEKAKSPVKEEAKSPQKEEAKSPAEVKSPEKAKSPVEAKSPEKAKSPVKEEAKSPEKAKSPEKEEAKSPEKAKSPEKAKSPVKEEAKSPVEAKSPEKAKSPVKEEAKSPEKAKSPVKEEAKSPEKAKSPVKEEAKSPEKAKSPVKEEAKSPEKAKSPVKEEAKSPEKAKSPVKEEAKSPEKAKSPVEAKSPEKAKSPAKEEAKSPEKAKSPVKEEAKSPEKAKSPVKEEAKSPEKAKSPAKEEAKSPEKAKSPEKPKSPVKEEAKSPEKPKSPVKEEAKSPEKPKSPVKEEAKSPEKAKSPVKDEAKAPGKEVLKKEEAKSPVKEEEKPQEVRAKEPPKKAEEEKAPAIPKAEEKKDSKKDEVPKKEAPKPEEKKEPAVEKPKEPKAEAKKEEAEDKKKATTPEKEAPAKGKEEAKPKEKTEVAKKEPDDAKPKEPSKAAEKPKKEETPAAPEKKDAKEGKAAEAKKPEEKPKTEAQAKEPGKEASTPGKAKAEKSSSTDQKDSRPAEKAAEDKATKGEK

SEQ ID NO: 11 - protein, 1064 aa, Rattus norvegicus
MMSFGGADALLGAPFAPLHGGGSLHYALSRKAGAGGTRSAAGSSSGFHSWARTSVSSVSASPSRFRGAGAASSTDSLDTLSNGPEGCVVAAVAARSEKEQLQALNDRFAGYIDKVRQLEAHNRSLEGEAAALRQQQAGRAAMGELYEREVREMRGAVLRLGAARGQLRLEQEHLLEDIAHVRQRLDEEARQREEAEAAARALARFAQEAEAARVELQKKAQALQEECGYLRRHHQEEVGELLGQIQGCGAAQAQAQAEARDALKCDVTSALREIRAQLEGHTVQSTLQSEEWFRVRLDRLSEAAKVNTDAMRSAQEEITEYRRQLQARTTELEALKSTKESLERQRSELEDRHQVDMASYQDAIQQLDNELRNTKWEMAAQLREYQDLLNVKMALDIEIAAYRKLLEGEECRIGFGPSPFSLTEGLPKIPSMSTHIKVKSEEKIKVVEKSEKETVIVEEQTEEIQVTEEVTEEEDKEAQGEEEEEAEEGGEEAATTSPPAEEAASPEKETKSPVKEEAKSPAEAKSPAEAKSPAEAKSPAEVKSPAVAKSPAEVKSPAEVKSPAEAKSPAEAKSPAEVKSPATVKSPGEAKSPAEAKSPAEVKSPVEAKSPAEAKSPASVKSPGEAKSPAEAKSPAEVKSPATVKSPVEAKSPAEVKSPVTVKSPAEAKSPVEVKSPASVKSPSEAKSPAGAKSPAEAKSPVVAKSPAEAKSPAEAKPPAEAKSPAEAKSPAEAKSPAEAKSPAEAKSPVEVKSPEKAKSPVKEGAKSLAEAKSPEKAKSPVKEEIKPPAEVKSPEKAKSPMKEEAKSPEKAKTLDVKSPEAKTPAKEEAKRPADIRSPEQVKSPAKEEAKSPEKEETRTEKVAPKKEEVKSPVEEVKAKEPPKKVEEEKTPATPKTEVKESKKDEAPKEAQKPKAEEKEPLTEKPKDSPGEAKKEEAKEKKAAAPEEETPAKLGVKEEAKPKEKAEDAKAKEPSKPSEKEKPKKEEVPAAPEKKDTKEEKTTESKKPEEKPKMEAKAKEEDKGLPQEPSKPKTEKAEKSSSTDQKDSQPSEKAPEDKAAKGDK

SEQ ID NO: 12 - protein, 1090 aa, Mus musculus
MMSFGSADALLGAPFAPLHGGGSLHYSLSRKAGPGGTRSAAGSSSGFHSWARTSVSSVSASPSRFRGAASSTDSLDTLSNGPEGCVVAAVAARSEKEQLQALNDRFAGYIDKVRQLEAHNRSLEGEAAALRQQQAGRAAMGELYEREVREMRGAVLRLGAARGQLRLEQEHLLEDIAHVRQRLDEEARQREEAEAAARALARFAQEAEAARVELQKKAQALQEECGYLRRHHQEEVGELLGQIQGCGAAQAQAQAEARDALKCDVTSALREIRAQLEGHAVQSTLQSEEWFRVRLDRLSEAAKVNTDAMRSAQEEITEYRRQLQARTTELEALKSTKESLERQRSELEDRHQADIASYQDAIQQLDSELRNTKWEMAAQLREYQDLLNVKMALDIEIAAYRKLLEGEECRIGFGPSPFSLTEGLPKIPSISTHIKVKSEEMIKVVEKSEKETVIVEGQTEEIRVTEGVTEEEDKEAQGQEGEEAEEGEEKEEEEGAAATSPPAEEAASPEKETKSRVKEEAKSPGEAKSPGEAKSPAEAKSPGEAKSPGEAKSPGEAKSPAEPKSPAEPKSPAEAKSPAEPKSPATVKSPGEAKSPSEAKSPAEAKSPAEAKSPAEAKSPAEAKSPAEAKSPAEAKSPATVKSPGEAKSPSEAKSPAEAKSPAEAKSPAEAKSPAEVKSPGEAKSPAEPKSPAEAKSPAEVKSPAEAKSPAEVKSPGEAKSPAAVKSPAEAKSPAAVKSPGEAKSPGEAKSPAEAKSPAEAKSPIEVKSPEKAKTPVKEGAKSPAEAKSPEKAKSPVKEDIKPPAEAKSPEKAKSPVKEGAKPPEKAKPLDVKSPEAQTPVQEEAKHPTDIRPPEQVKSPAKEKAKSPEKEEAKTSEKVAPKKEEVKSPVKEEVKAKEPPKKVEEEKTLPTPKTEAKESKKDEAPKEAPKPKVEEKKETPTEKPKDSTAEAKKEEAGEKKKAVASEEETPAKLGVKEEAKPKEKTETTKTEAEDTKAKEPSKPTETEKPKKEEMPAAPEKKDTKEEKTTESRKPEEKPKMEAKVKEDDKSLSKEPSKPKTEKAEKSSSTDQKESQPPEKTTEDKATKGEK

SEQ ID NO: 13 - protein, 890 aa, Gallus gallus
MRNTAGCQGPAPGPPSTPSGLRRRLGAVPLPVPVPVPVPAERRGGPDGPYKAGGGGSGAAVRAGATMSLVLEALLVPPGGLRKEPSRLAPRSAASSSGFHSWPGPPGTVGRARGGGGGSAAASSTESLDSLNGEPRARNEKELLQVLNDRFAGYIERVRALEGQNRALAAEAAALRGQQAGRSAMGELAQQVRLDKLSEVAKVNTDAMRVAQEEISEYRRQLQSKTTELEALKGTQESLERQRQDSEERHHADVLSYQETIQQLDNELRNTKWEMAAQLREYQDLLNVKMALDIEIAAYRKLLEGEEYRIETGFGMVSYPEVVPKAPSIAANIKVKSEEKIKVVEKSEKETVIVEEQTEEIQVTEEVTEEEEAEKEAEEEKAEEEKEEEKAEPEGEEEAKSPEKPESPTKEEAKSPAVKSPEKPSTPSKEEAKSPTVKSPEKPSTPSKEEAKSPTVKSPEKPSTPSKEEDKSPAVKSPEKPSTPSKEEAKSPAVKSPEKPSTPSKEEAKSPAVKSPEKPAPPSKEEAKSPASPEKPAPPSKEEAKSPAVKSPEKPPVPSKEEAKSPPVKSPEKPATPLKEEAKTPTVKSPEKPPTPTKEETKPPSVKSPEKPPTALKEEAKSPAVKSPEKPATPSKEEAKSPAVKSPEKPSTPPKEEAKTPAVKSPEKPTPPSKEEAKTPAAKSPEKPVSPSKEEAKTPTVKSPEKAKSPAKEEAKSPQKEAAPAKEPVPAPPKEPKAPAKEEQPKEEKAPPKPAAEGRKDEAPKKDAPAKAEEKPKEKAAAGPEPPAPQVKETKAASKPAEEGKAEKDAQPKPQQEGGKAAVKEPEKPKAEEKVEEPKKTVEEPKKEKVEDPKSKPKDDSKASKEPPKAEKPPSKEGKAPEPAPAVGKK

SEQ ID NO: 14 - protein, 551 aa, Homo sapiens
KEAKEEEGKEEEGGEEEEAEGGEEETKSPPAEEAASPEKEAKSPVKEEAKSPAEAKSPEKEEAKSPAEVKSPEKAKSPAKEEAKSPPEAKSPEKEEAKSPAEVKSPEKAKSPAKEEAKSPAEAKSPEKAKSPVKEEAKSPAEAKSPVKEEAKSPAEVKSPEKAKSPTKEEAKSPEKAKSPEKAKSPEKEEAKSPEKAKSPVKAEAKSPEKAKSPVKAEAKSPEKAKSPVKEEAKSPEKAKSPVKEEAKSPEKAKSPVKEEAKTPEKAKSPVKEEAKSPEKAKSPEKAKTLDVKSPEAKTPAKEEARSPADKFPEKAKSPVKEEVKSPEKAKSPLKEDAKAPEKEIPKKEEVKSPVKEEEKPQEVKVKEPPKKAEEEKAPATPKTEEKKDSKKEEAPKKEAPKPKVEEKKEPAVEKPKESKVEAKKEEAEDKKKVPTPEKEAPAKVEVKEDAKPKEKTEVAKKEPDDAKAKEPSKPAEKKEAAPEKKDTKEEKAKKPEEKPKTEAKAKEDDKTLSKEPSKPKAEKAEKSSSTDQKDSKPPEKATEDKAAKGK

SEQ ID NO: 15 - protein, 510 aa, Homo sapiens
KEAKEEEGKEEEGGEEEEAEGGEEETKSPPAEEAASPEKEAKSPVKEEAKSPAEAKSPEKEEAKSPAEVKSPEKAKSPAKEEAKSPPEAKSPEKEEAKSPAEVKSPEKAKSPAKEEAKSPAEAKSPEKAKSPVKEEAKSPAEAKSPVKEEAKSPAEVKSPEKAKSPTKEEAKSPEKAKSPEKAKSPEKEEAKSPEKAKSPVKAEAKSPEKAKSPVKAEAKSPEKAKSPVKEEAKSPEKAKSPVKEEAKSPEKAKSPVKEEAKTPEKAKSPVKEEAKSPEKAKSPEKAKTLDVKSPEAKTPAKEEARSPADKFPEKAKSPVKEEVKSPEKAKSPLKEDAKAPEKEIPKKEEVKSPVKEEEKPQEVKVKEPPKKAEEEKAPATPKTEEKKDSKKEEAPKKEAPKPKVEEKKEPAVEKPKESKVEAKKEEAEDKKKVPTPEKEAPAKVEVKEDAKPKEKTEVAKKEPDDAKAKEPSKPAEKKEAAPEKKDTKEEKAKKPEEKPKTEAKAKEDD

(19) United States
(12) Patent Application Publication
(10) Pub. No.: US 2014/0038834 A1
(43) Pub. Date: Feb. 6, 2014

(54) NOVEL BIOMARKERS FOR DETECTING NEURONAL LOSS

(71) Applicant: Axel Franz Siegfried Petzold, Amsterdam (NL)

(72) Inventor: Axel Franz Siegfried Petzold, Amsterdam (NL)

(73) Assignee: VERENIGING VOOR CHRISTELIJK HOGER ONDERWIJS, WETENSCHAPPELIJK ONDERZOEK EN PATIËNTENZORG, Amsterdam (NL)

(21) Appl. No.: 13/808,758

(22) PCT Filed: Jul. 7, 2011
(86) PCT No.: PCT/NL2011/050496

§ 371 Date: May 23, 2013

Related U.S. Application Data
(60) Provisional application No. 61/361,954, filed on Jul. 7, 2010.

(30) Foreign Application Priority Data
Jul. 7, 2010 (EP) 10168774.7

(51) Int. Cl. G01N 33/68

(52) U.S. Cl. CPC G01N 33/6893
USPC 506/9; 435/7.1; 435/7.9; 435/7.92; 435/23; 435/68.1; 530/324; 530/326; 530/350; 530/387.9

(57) ABSTRACT

The present invention relates to a biomarker for the detection of brain damage or a disease associated with loss of neurons, said biomarker comprising a protein fragment of the neurofilament heavy chain (NfH) protein in a biological sample, wherein said protein fragment is a polypeptide selected from the group consisting of i) a polypeptide having the amino acid sequence selected from the group consisting of SEQ ID NO:1, SEQ ID NO:2, SEQ ID NO:3, SEQ ID NO:4, SEQ ID NO:5, and polypeptides composed of amino acids 476-1026 and 476-986 of SEQ ID NO 6; ii) a protein fragment of the NfH protein having an amino acid sequence that is at least 60% identical to SEQ ID NO:1, at least 60% identical to SEQ ID NO:2, at least 60% identical to SEQ ID NO:3, at least 60% identical to SEQ ID NO:4, at least 60% identical to SEQ ID NO:5; or at least 60% identical to a polypeptide composed of amino acids 476-1026 and.or 476-986 of SEQ ID NO 6; iii) an Enterokinase cleavage product of the NfH protein; and iv) a polypeptide that is derived from a naturally occurring allelic variant of the nucleic acid coding for NfH protein.

### FIELD OF THE INVENTION

The invention is in the field of medical diagnostics. More in particular, the invention relates to diagnosis of neurological damage, notably detection of loss of neurons and axons. The invention provides biomarkers for detecting loss of neurons and axons, and means and methods for the diagnostic and prognostic use of these biomarkers in monitoring, diagnosis and prognosis of neurological disorders, in particular antibodies, protein standards and kits-of-parts.

### BACKGROUND OF THE INVENTION

Neurons are the cells belonging to the nervous system and are responsible for the transfer of electrical and chemical signals to different types of cells in the body. Loss of these cells is irreversible, as differentiated neurons are generally not capable of dividing. Hence, neurons which are dead due to disease, physical or chemical damage are not replaced by new neurons. The loss of neurons may cause disability. The range of disabilities is wide, embracing cognitive deficits, mood disorders, problems with locomotion, impaired co-ordination, loss of balance, pain and disabilities caused by the loss of afferent pathways (e.g. blindness, deafness). There are many diseases which are caused by, or associated with the loss of neurons. Treatment of diseases associated with neuronal loss is costly. Therefore, accurate diagnosis and prognosis of such diseases is important. For this reason, there has been extensive research into identifying suitable biomarkers which can detect the loss of neurons. Until now this has not resulted in a test suitable for the quantification of loss of neurons from patient's samples.

A number of biomarkers associated with neuronal loss have been implicated. Among these is a group of proteins known as neurofilaments (Nf) which are highly specific for the neuro-axonal compartment. This group of Nf contains at least four proteins named NfL, NfM, NfH, alpha-internexin and in the case of the peripheral nervous system also peripherin.

These biomarkers have drawbacks. The polymers of the Nf proteins require rigorous sample handling which reduces their application as biomarkers. None of these subunits is suitable as a biomarker. The main drawback of NfL is that it is susceptible to proteases, which makes it prone to degradation, particularly in the protease rich cerebrospinal fluid, resulting in inaccurate measurements. To overcome this problem, rigorous sample handling, i.e. snap freezing of cerebrospinal fluid, is required. This makes the application of NfL as a biomarker difficult. The NfH protein has also long been considered as a potential biomarker, but the large molecular size, aggregate formation and low water solubility of this protein are obstacles for its use as a biomarker. The presence of NfL in protein aggregates may result in shielding of epitopes. In addition, the presence of NfL in protein aggregates has led to the observation that dilution of a sample comprising these aggregates results in an apparent increase in the level of free protein due to an increased release of NfL from the aggregate in the dilution (Lu et al. 2010, Journal of Neuroscience Methods 195: 143-150). This effect, termed hook-effect, further hampers the use of NfL as a quantitative biomarker.

It is an objective of the invention to provide a quantitative biomarker which correlates well with neuronal loss, is stable, does not form aggregates, is not susceptible to a hook-effect, is small enough to be suitable for sampling by medical devices such as microdialysis, and is water soluble.

### SUMMARY OF THE INVENTION

The invention is based on the discovery of a novel neuronal proteolytic pathway. The present inventors observed a predominant neuro-axonal expression of Pavlov's enterokinase (EK, a serine protease which is also known as enteropeptidase, Enzyme Nomenclature: EC 3.4.21.9). Enterokinase K is membrane bound and cleaves the neurofilament heavy chain (NfH) at different positions, including at position 476 and 986. Using a 100 kDa microdialysis cutoff membrane two proteolytic breakdown products, eNfH (NfH476-986 and NfH476-1026) could be quantified with a relative recovery of 20% in biological samples from patients suffering from diseases associated with neuronal loss or brain damage.

The invention provides a biomarker for the detection of brain damage or a disease associated with loss of neurons comprising a protein fragment of the neurofilament heavy chain (NfH) protein in a biological sample, wherein said protein fragment is a polypeptide selected from the group consisting of a polypeptide having or consisting of the amino acid sequence selected from the group consisting of SEQ ID NO:1, SEQ ID NO:2, SEQ ID NO:3, SEQ ID NO:4 and SEQ ID NO:5, more preferably of SEQ ID NO:2, SEQ ID NO:3, SEQ ID NO:4 and SEQ ID NO:5, most preferably the polypeptides composed of amino acids 476-1026 (SEQ ID NO:14) and 476-986 (SEQ ID NO:15) of SEQ ID NO 6, a protein fragment of the NfH protein having the amino acid sequence that is at least 60% identical to SEQ ID NO:1, at least 60% identical to SEQ ID NO:2, at least 60% identical to SEQ ID NO:3, at least 60% identical to SEQ ID NO:4 or at least 60% identical to SEQ ID NO:5, an Enterokinase cleavage product of the NfH protein; and a polypeptide that is a encoded by a naturally occurring allelic variant of the nucleic acid coding for a protein having or consisting of the amino acid sequence selected from the group consisting of SEQ ID NO:1, SEQ ID NO:2, SEQ ID NO:3, SEQ ID NO:4 and SEQ ID NO:5, more preferably of SEQ ID NO:2, SEQ ID NO:3, SEQ ID NO:4 and SEQ ID NO:5, most preferably the polypeptides composed of amino acids 476-1026 (SEQ ID NO:14) and 476-986 (SEQ ID NO:15) of SEQ ID NO 6.

Preferably, said protein fragment is an Enterokinase cleavage product having a molecular weight selected from the group consisting of 101-112 kDa or 94-104 kDa, 89-99 kDa, 35-39 kDa and 25-28 kDa, more preferably of 101-112 kDa and 94-104 kDa, as determined by migration in gel-electrophoresis.

Said biomarker is preferably a polypeptide selected from the group consisting of a polypeptide with a calculated molecular weight of about 54 kDa having the amino acid sequence of SEQ ID NO:1, a polypeptide with a calculated molecular weight of about 39 kDa having the amino acid sequence of SEQ ID NO:2, a polypeptide with a calculated molecular weight of about 2 kDa having the amino acid sequence of SEQ ID NO:3, a polypeptide with a calculated molecular weight of about 15 kDa having the amino acid sequence of SEQ ID NO:4, a polypeptide with a calculated molecular weight of about 4 kDa having the amino acid sequence of SEQ ID NO:5, a polypeptide with a calculated molecular weight of about 60 kDa having the amino acid sequence of SEQ ID NO:14, a polypeptide with a calculated molecular weight of about 56 kDa composed of the amino acid sequence of SEQ ID NO 15, a polypeptide with a calculated molecular weight of about 41 kDa composed of amino acids 476-852 of SEQ ID NO 6, a polypeptide of about 21 kDa composed of amino acids 835-1026 of SEQ ID NO 6, a polypeptide of about 17 kDa composed of amino acids 835-986 of SEQ ID NO 6, a polypeptide of about 19 kDa composed of amino acids 852-1026 of SEQ ID NO 6, a polypeptide of about 92 kDa composed of amino acids 1-835 of SEQ ID NO 6, a polypeptide of about 94 kDa composed of amino acids 1-852 of SEQ ID NO 6, a polypeptide of about 109 kDa composed of amino acids 1-986 of SEQ ID NO 6, a polypeptide that is at least 60% identical to the indicated polypeptides and/or that are encoded by a naturally occurring allelic variant of the nucleic acid coding for NfH.

The invention further provides a method for preparing a protein fragment as defined herein, comprising steps of combining a NfH protein and Enterokinase in an aqueous solution, allowing the cleavage of said NfH protein; and preferably isolating the resulting protein fragment.

The invention further provides an in vitro method for determining the level of a biomarker for brain damage or a disease associated with loss of neurons in a suspect, comprising steps of detecting in a biological sample of said subject a biomarker according to the invention, comparing the amount of said biomarker in said biological sample with a reference value. Said method is preferably used for monitoring, diagnosis and/or prognosis of brain damage or of a disease associated with a loss of neurons in a subject or animal or cell-culture system. The method may further comprise determining whether loss of neurons has occurred or determining the level of loss of neurons based on the comparison to reference values or in the case of serial sampling to the subjects/animal/cell-culture own baseline values. In a preferred embodiment, an elevated level of said biomarker or a rise of said biomarker from baseline is indicative of the presence of said brain damage or a disease associated with a loss of neurons in the case of diagnosis or indicative of the progression of said brain damage or a disease associated with a loss of neurons in the case of prognosis or result of an experimental procedure in an animal experiment or cell-culture. Preferably, said reference values are values for healthy subjects in the case of diagnosis, or values of the same subject at earlier points in time in the case of prognosis. Preferably, said biological sample is a sample selected from the group consisting of cortical brain tissue, blood, plasma, serum, cerebrospinal fluid, amniotic fluid, urine, vitreous body or any type of tissue homogenate containing neurons or axons. Said biological sample is preferably pre-delivered. Preferably, said biological sample is a microdialysate of the extracellular fluid of the brain, preferably obtained using a microdialysis membrane having a large enough pore size. Preferably, detecting said biomarker is detected by quantitative or qualitative methods, by ELISA, luminex or ECL based technology, gel-electrophoresis, nephelometry, mass spectroscopy, any variation of dipsticks or enzymatic reactions. For instance, the method may comprise exposing said biological sample or a purified fraction thereof to a specific antibody or a capture agent to allow said biomarker to form a complex with said specific antibody and detecting said complex. Alternatively the biological sample or a purified fraction thereof may be subjected to an ionizing treatment to allow said protein fragment to generate charged molecules or molecule fragments, measuring the mass-to-charge ratio of said charged molecules or molecule fragments and identifying the biomarker based on the measured mass-to-charge ratio.

In preferred embodiments of aspects of the invention the disease is a neurodegenerative disease. This includes primary/inherited and/or secondary/acquired forms of neurodegeneration. Specifically the invention can be used to monitor and/or diagnose neurotoxic effects of medication and other medical interventions such as radiation. More specifically, a biomarker according to the invention can be used as a safety marker for treatment and treatment trials. In alternative preferred embodiments, said brain damage is traumatic brain injury (TBI), preferably TBI with suspected diffuse axonal injury (DAT). The term TBI also includes diffuse neuronal damage in the context of blast TBI such as occurring in military action or by terrorist acts and/or major incidents resulting in civil causalities. Preferably, said biological sample is collected within a week, preferably within 12 hours after the onset of said brain damage or a disease associated with a loss of neurons.

Further provided is a method for preparing a biomarker according to the invention comprising steps of combining a NfH protein and Enterokinase in an aqueous solution, allowing the cleavage of said NfH protein, and optionally isolating the resulting protein fragment.

Further provided is a biomarker that is prepared according to a method of the invention.

The invention further provides the use of a protein as defined herein as a reference protein in a test for detecting neuronal loss in a sample of a subject, animal or cell-culture.

Further provided is an antibody or a molecule having the antigen-binding portion of an antibody which specifically detects a biomarker according to the invention.

The invention further provides a kit of parts comprising a protein as defined herein and/or specific antibodies according to the invention.

### DESCRIPTION OF THE DRAWINGS

FIG. 1 illustrates that neurofilaments are a protein biomarkers for neuronal loss. (A) The cytoskeleton of the neuron and axon contains the neurofilament heavy chain (NfH, filled red boxes). The number of laser-captured neurons/axons relates to the NfH levels quantified by enzyme-linked immunosorbent assay. (B) The estimated quantitative relationship between the number of laser-captured PC12 cells and the NfH concentration is expressed as: (number of PC12 cells˜1245×NfH−46, R=0.98, P=0.013). The lowest number of laser-captured cells giving for quantifiable NfH levels was 50 (data not shown). (C) The percentage of NfH to total soluble protein is 24.6 times higher in the human grey matter compared with PC12 cells. (D) In the injured human brain, NfH is released from the neuronal compartment into the extracellular fluid (ECF, dark grey box). Placement of a microdialysis catheter into the human cortex, adjacent to neurons, allows recovery of NfH from the human extracellular fluid. (E) The in vitro recovery experiment showed that NfH could be recovered from a source solute using a 100 kDa microdialysis catheter, but not a 20 kDa catheter. (F) Consequently, the relative recovery of NfH was very low for the 20 kDa catheter and 20% for the 100 kDa microdialysis catheter.

FIG. 2 illustrates the identification of five new NfH enterokinase cleavage products. (A) Gel electrophoresis of pooled extracellular fluid (ECF) (4-12% Bis-Tris gel, Coomassie stain). There are a number of distinct bands visible between 98 and 188 kDa in extracellular fluid pooled (100 kDa catheter) from samples with high NfH, which can not be visualized from the extracellular fluid of samples with non-measurable NfH. (B) The immunoblot shows a ˜100 kDa protein recognized by SMI34 in the extracellular fluid pooled from samples with a high NfH concentration (Lanes 2 and 4) but not from samples with non-measurable NfH levels (Lane 3). No other higher molecular weight NfH fragments were recovered through the 100 kDa microdialysis membrane (3-8% Tris-acetate gel, because of overexposure of the molecular weight markers (MW), the gain for Lane 1 was digitally reduced). (C) The ˜100 kDa protein fragment is recognized by monoclonal antibodies against phosphorylated (SMI34, SMI35) and non-phosphorylated (SMI32, SMI38, SMI37, SMI311) NfH epitopes. The strongest immunoreactivity was observed with SMI34 and SMI38. There was no binding to the detecting rabbit anti-mouse horseradish peroxidase labelled antibody (RaM-HRP). Equally, there was no binding with monoclonal antibodies against NfL or NfM, known to have a small degree of cross-reactivity with the NfH head/rod region. This suggests that the recovered protein stems from the NfH tail region (4-12% Bis-Tris, 1 well gel, antibody incubation in manifold). (D) Immunoreactivity against hyperphosphorylated NfH (SMI34). The 100-120 kDa band from human extracellular fluid (Lane 2) migrates in a similar range to a fraction present in HPLC-purified bovine NfH (Lane 3), which can be further enhanced by limited proteolysis with enterokinase (EK, Lane 4) but not α-chymotrypsin (CT, Lane 4). Not all of the purified higher molecular weight NfH migrates into the gel (top part of Lanes 3-4). There is no immunoreactivity of SMI34 with dephosphorylated purified or proteolysed NfH (Lanes 7-9). (E) Immunoreactivity against non-phosphorylated NfH (SMI38). The 100-120 kDa band from human extracellular fluid (Lane 2) migrates in the same range as partially enterokinase proteolysed non-phosphorylated NfH (Lane 8). Enterokinase proteolysis reveals three additional bands (˜90, 40, 20 kDa) for both phosphorylated (Lane 4) and non-phosphorylated NfH (Lane 8). (F) Cleavage of NfH with enterokinase occurs at four sites (ExPASy, peptide cutter), leaving 12 hypothetical proteolytic fragments, five of which probably explain the immunoreactivity against SMI38 (Lanes 4 and 8 in E). Cleavage at position 476 alone (first dark grey box) or positions 476 and 986 (second dark grey box) is thought to produce the polypeptides giving rise to the immunoreactivity against SMI34 and SMI38 seen in the 100-120 kDa range (Lanes 1 and 3 in D, Lanes 1 and 8 in E). The immunoreactivity of the phosphorylated NfH 35-39 kDa and 25-28 kDa fragments (bottom two light grey boxes) with the non-phosphoepitope specific monoclonal antibody SMI38 (Lane 4 in E) may be explained by structure related dephosphorylation of a serine residue (e.g. at position 865), adjacent to the enterokinase cleavage sites or alternatively by limited phosphatase activity of enterokinase. ALP=alkaline phosphatase.

FIG. 3 illustrates that Enterokinase is expressed in cortical neurons and axons. (A) Immunohistochemistry demonstrates that cortical neurons and their axons are selectively stained for enterokinase (frontal lobe, traumatic brain injury). Enterokinase appears to localize to the membrane of neurons and axons (scale bar=10 μm). (B) A similar staining pattern for enterokinase is seen in control tissue (frontal lobe, epilepsy). This suggests that enterokinase is expressed in a subset of human cortical neurons and not a specific phenomenon seen in traumatic brain injury (scale bar=10 μm). (C) Confocal imaging shows that large neurons positive for enterokinase also stain for phosphorylated NfH in traumatic brain injury (scale bar=10 μm). (D) Enterokinase was not found in neurons, which predominantly expressed dephosphorylated NfH SMI32 in traumatic brain injury (scale bar=25 μm). (E) The immunoblot shows presence of the heavy chain of enterokinase in homogenated human cortex (four traumatic brain injury cases, Lanes 4, 6, 8 and 10), but not in adjacent white matter tissue (Lanes 3, 5, 7 and 9). There is a mild degree of cross-reactivity of the antibody (LC13) with the enterokinase light chain, seen for the purified protein only (Lane 2, ˜50 kDa). The molecular weight markers are shown in Lane 1.

FIG. 4 shows apoptosis of cortical neurons in traumatic brain injury. (A) Caspase-3 positive pyramidal cells in the vicinity of a cortical contusion from a patient with traumatic brain injury (cases 18-95). (B) Caspase-3 positive neuronal like cells with dendritic processes in the vicinity of the contusion area of another patient with traumatic brain injury. Scale bar=10 μm.

FIG. 5 shows extracellular fluid NfH levels in traumatic brain injury. The longitudinal profile of extracellular fluid NfH levels in individual patients is shown in relation to catheter localization, time from catheter insertion and time from injury. The tip of the catheter is indicated by a black arrow overlaid to the patients' CT brain scan. Early extracellular fluid NfH peaks are identified by single arrows. Secondary extracellular fluid NfH peaks occurring after a period of decreasing or consistently low extracellular fluid NfH levels are indicated by double arrows. Note that the scale for the y-axis is individually optimized for best visualization of extracellular fluid NfH levels.

FIG. 6 shows that multimodal monitoring illustrates that extracellular fluid NfH levels peaked in the acute phase of traumatic brain injury coinciding with derangement of physiological parameters. The mode of injury in patients with traumatic brain injury was either due to a fall (dotted line) or high-velocity impact traumatic brain injury (closed line). The multimodal data are plotted over time using 3 h epochs. (A) Extracellular fluid NfH levels were significantly higher in high impact traumatic brain injury compared with a fall (P<0.0001). (B) The extracellular fluid lactate to pyruvate ratio was highest in high-velocity impact traumatic brain injury (P<0.0001) suggesting critical energy demand under anaerobic metabolic conditions. (C) The brain tissue oxygenation (BtO2) did not differ significantly between groups. (D) Initially the brain temperature (BT) was significantly higher in high-velocity impact traumatic brain injury (P<0.0001), but a crossover of the curves occurred after six epochs, reflecting active cooling as part of intra-cranial pressure targeted management. (E) The systemic oxygen saturation (SpO2) remained in the target range (>98%) throughout and did not differ significantly between the groups. (F) There were no periods of systemic hypotension. The mean arterial blood pressure (ABP) increased to >80 mmHg after nine epochs due to volume resuscitation and inotropic support as part of targeted intra-cranial pressure management. (G) The mean intra-cranial pressure (ICP) was significantly higher after a fall (P<0.0001) and continued to increase. The critical threshold of 20 mmHg was passed after the 10th epoch. (H) Successful maintenance of the cerebral perfusion pressure (CPP) above 60 mmHg was achieved by increasing the mean arterial blood pressure until Epoch 15. From there on the continuous increase of intra-cranial pressure in patients with a fall started to affect the cerebral perfusion pressure. The means±SD are shown.

FIG. 7 shows Mechanisms leading to neuronal loss and increase of extracellular fluid NfH. (A) The strength of significant correlations (Spearman's R, y-axis) between extracellular fluid NfH (eNfH) levels and physiological parameters during 3 h epochs (x-axis) is shown. The lactate to pyruvate ratio (LPR) is indicated by black bars, arterial blood pressure (ABP) by dark grey bars, cerebral perfusion pressure (CPP) by light green bars, brain tissue oxygenation (BtO2) by hatched bars, brain temperature (BT) by blue bars. The grey box overlay highlights the correlations for which the raw data are shown. (B) The lactate to pyruvate ratio correlated with extracellular fluid NfH levels (Epoch 2). (C) Because the lactate to pyruvate ratio increases under anaerobic metabolic conditions, this suggests that the resulting metabolic penalty may cause neuronal loss with subsequent increase of extracellular fluid NfH. (D) The mean arterial blood pressure correlated inversely with extracellular fluid NfH levels. (E) This raises the question whether ischaemic neuronal injury may be reduced by more aggressive arterial blood pressure management. (F) The brain tissue oxygenation correlated with extracellular fluid NfH levels. (G) This suggests that increasing BtO2 may saturate systems of endogenous protection. The resulting O₂ toxicity may cause neuronal injury resulting in increased levels of extracellular fluid NfH. (H) The brain temperature correlated with extracellular fluid NfH levels. (I) This suggests that higher brain temperature may accelerate mechanisms leading to neuronal apoptosis resulting in an increase of extracellular fluid NfH levels. ROS=reactive oxygen species, ***P<0.0001, **P<0.001, *P<0.01, †P<0.05.

### DETAILED DESCRIPTION OF THE INVENTION

### Definitions

The term “neurofilament” refers to the intermediate filament family containing: L, M, H, alpha-internexin and in case of the peripheral nervous system also peripherin. Neurofilaments are found in vertebrate neurons in especially high concentrations along the axons, where they appear to regulate axonal diameter. Neurofilaments are proteins which are specific for cells of the central nervous system (CNS). NfH has an functions in axons that are not subserved by the two smaller NF proteins in particular with regard to protein-protein interaction and binding to cell organelles such as mitochondria.

The term “NfH” refers to the neurofilament heavy chain protein. The human NfH (SEQ ID NO:6) is coded on chromosome 22q12.2 by the NfH gene (NC_{—}000022.10 or NT_{—}011520.12) and consists of 1020 amino acids. The molecular mass of the amino acids corresponds to 111 kDa. One may also refer to the molecular mass derived from SDS gels which are influenced by the charge/weight of bound phosphates and therefore the molecular mass ranges from 190 to 210 kDa for the various phosphoforms. The term NfH as used herein refers to an NfH protein of any species. Therefore, any NfH protein encoded by an ortholog of the human NfH may be used in aspects of the invention. Several NfH orthologs are known and the amino acid sequences of some are provided in the sequence listing.

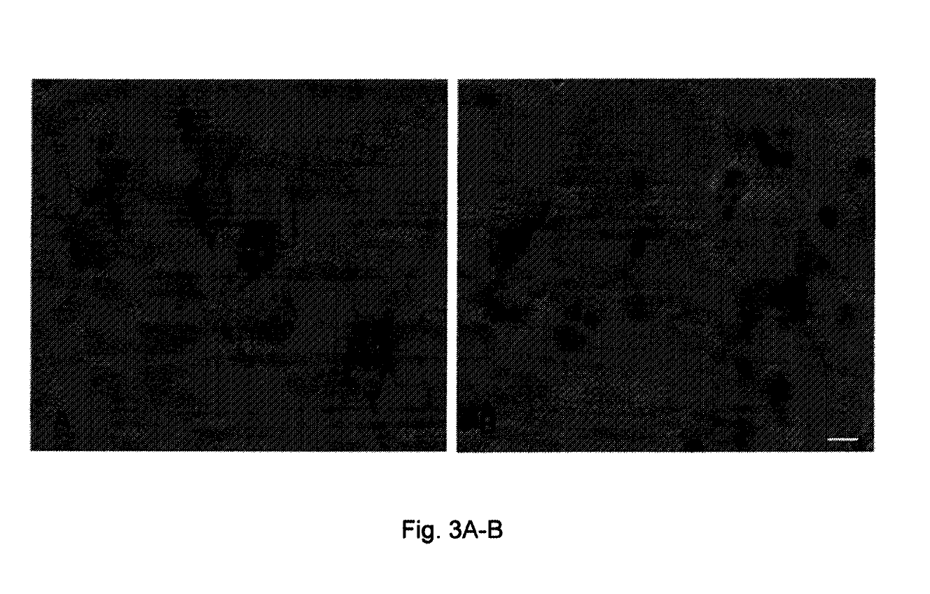

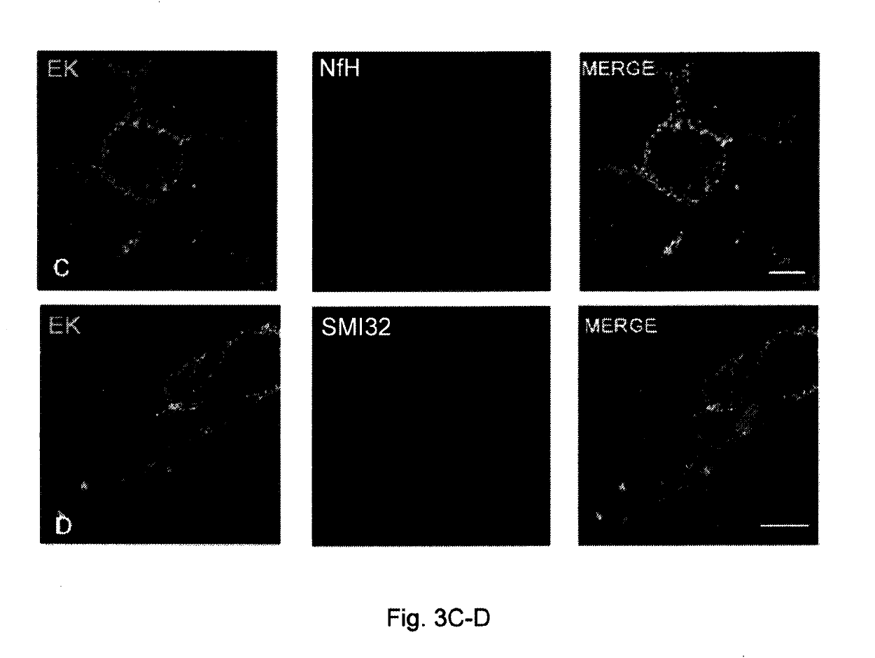

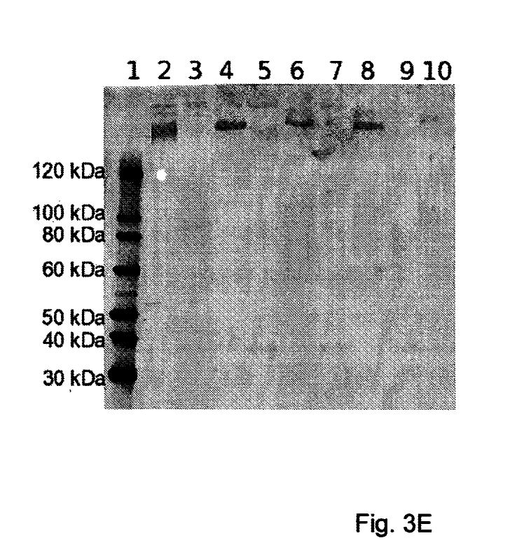

The terms “eNfH”, “eNfH polypeptide”, “eNfH protein fragment” and “eNfH fragment” are used interchangeably herein and refer to a proteolytic breakdown product of the neurofilament heavy chain protein (NfH) catalysed by Enterokinase (EK). In the human NfH protein, four EK cleavage sites have been detected, as shown in FIG. 3. Cleavage of the human NfH results in 12 different cleavage products (eNfH), of which 5 are shown in FIG. 3.

The term “Enterokinase K” or short “Enterokinase” as used herein refers to a specific protease of which the human form cleaves after lysine at its cleavage site Asp-Asp-Asp-Asp-Lys. It will sometimes cleave at other basic residues, depending on the conformation of the protein substrate. In the case of digestion of the human NfH protein, four EK cleavage sites have been detected including Glu-Glu-Glu-Glu-Lys, Glu-Glu-Glu-Lys and Glu-Asp-Asp-Lys. The Enterokinase as referred to herein may in principle be of any species. Enterokinases of other species than human may be specific for other cleavage sites. Different Enterokinases are described in Boulware, K. T. and Daugherty, P. S. (2006) Proc Natl Acad Sci USA, 103(20):7583-7588. The gene sequence of the human enterokinase gene is described in Kitamoto Y et al., Biochemistry. 1995 Apr. 11; 34(14):4562-8. The amino acid sequence of the human Enterokinase protein is provided herein as SEQ ID NO: 7. Preferably, said Enterokinase is a protein having the amino acid sequence which is at least 90, 91, 92, 93, 94, 95, 96, 97, 98, 99% identical to SEQ ID NO:7. Generally, 0.00016 ug of Enterokinase will cleave 25 ug of test substrate to 95% completion in 16 hours or less at 25° C. 1 IU of Enterokinase will cleave 50 ug of test substrate to 95% completion in 16 hours or less at 25° C., whereby the Unit Assay Conditions are 20 mM Tris-HCl (pH 8.0 @ 25° C.), 50 mM NaCl, 2 mM CaCl₂, 25 ug of an MBP fusion protein test substrate and enzyme. Incubate at 23° C. Hence, appropriate reaction conditions can be inferred by one of skill in the art from the fact that an amount of enzyme required to cleave a fusion protein in a 16 hour reaction at room temperature ranges from 0.0001% to 0.5% (w/w). Cleavage of an MBP-paramyosin-ΔSal fusion protein with an enterokinase site requires 0.0006% of enzyme under Unit Assay Conditions of 20 mM Tris-HCl (pH 8.0 @ 25° C.), 50 mM NaCl, 2 mM CaCl₂, 25 ug of MBP-paramyosin-ΔSal fusion protein test substrate and enzyme with incubation at 23° C.

The term “reference protein” refers to a protein used in an assay as a control molecule. Preferably, said reference protein is used as a positive control. A positive control is used in a procedure to determine the nature or the amount of a detection signal in an assay known to give a positive result. A positive control is preferably used in a known amount and detected under the same or comparable conditions as the unknown amount of the same or comparable molecule to be detected.

The term “amino acid sequence similarity” as used herein denotes the presence of similarity between two proteins, fragments or polypeptides. Proteins have “similar” or “identical” sequences if the sequence of amino acids in the two sequences is the same when aligned for maximum correspondence. Sequence comparison between two proteins is generally performed by comparing portions of the two sequences over a comparison window to identify and compare local regions of sequence similarity. The comparison window is typically from about 10 to 80 contiguous amino acids. The “percentage of sequence similarity” and “percentage of identical amino acid sequence” for proteins, such as 50, 60, 70, 80, 90, 95, 98, 99 or 100 percent sequence similarity or identity may be determined by comparing two optimally aligned sequences over a comparison window, wherein the portion of the protein sequence in the comparison window may include amino acid deletions, modification or addition of single amino acids or groups of amino acids as compared to the reference sequence (which does not comprise additions or deletions) for optimal alignment of the two sequences. The percentage is calculated by: (a) determining the number of positions at which the identical amino acid occurs in both sequences to yield the number of matched positions; (b) dividing the number of matched positions by the total number of positions in the window of comparison; and (c) multiplying the result by 100 to yield the percentage of sequence similarity. Optimal alignment of sequences for comparison may be conducted by computerized implementations of known algorithms, or by visual inspection. Sequence comparison and multiple sequence alignment algorithms are readily available on the internet, for instance William Pearson's “LALIGN” program. The LALIGN program implements the algorithm of Huang and Miller, published in Adv. Appl. Math. (1991) 12:337-357. It can be found at http://www.ch.embnet.org/software/LALIGN_form.html.

The term “neuron” refers to a nerve cell. A neuron may be any of the conducting cells of the nervous system, consisting of a cell body, containing the nucleus and its surrounding cytoplasm, and the axon and dendrites.

The terms “loss of neurons” and “neuronal loss” as used herein are used interchangeably herein and refer to the process of irreversible and functional loss of a neuron. During this process, neurons are damaged and can no longer exert their function of transmitting information by electrical and/or chemical signalling. Loss of neurons is inexorably followed by loss of their axons.

The term “disease associated with a loss of neurons” refers to a neurological disorder wherein at least a symptom of such associated by or attributed to the loss of neurons manifests at some point in time during the disease. Symptoms may develop immediately after onset, for example after a major traumatic injury of the brain, but in some cases, such as in patients suffering from MS, symptoms may develop gradually. Herein, distinction is made between neurodegenerative diseases, which are associated with a progressive loss of neurons, typically as a result of subcellular processes and brain injuries or brain damage, which is mostly caused by physical damage.

The term “neurodegeneration” as used herein is the umbrella term for the progressive loss of structure or function of neurons, including death of neurons. In certain preferred embodiments, said disease is a neurodegenerative disease, which is caused by neurodegeneration. Neurodegerative diseases include: any form of optic neuritis, glaucoma, HIV, AIDS dementia complex, adrenoleukodystrophy, Alexander disease, Alpers' disease, Alzheimer's disease, amyotrophic lateral sclerosis, ataxia telangiectasia, Batten disease, bovine spongiform encephalopathy, Canavan disease, cerebrovascular pathology, Charcot-Marie-Tooth disease, corticobasal degeneration, Creutzfeldt-Jakob disease, dementia, dementia with Lewy bodies, diabetic neuropathy, diffuse myelinoclastic sclerosis, fatal familial insomnia, frontotemporal lobar degeneration, giant axonal neuropathy, glaucoma, Huntington's disease, Kennedy's disease, Krabbe disease, leprosy, Lyme disease, Machado-Joseph disease, malaria, multiple sclerosis, multiple system atrophy, neuroacanthocytosis, neuropathy, Niemann-Pick disease, neurofilament inclusion body dementia, neuromyelitis optica, Parkinson's disease, variants of classical Parkinson's disease, Pick's disease, primary lateral sclerosis, progressive supranuclear palsy, Refsum's disease, Sandhoff disease, spinocerebellar ataxia, stroke, subacute combined degeneration of spinal cord, Tabes dorsalis, Tay-Sachs disease, toxic encephalopathy, transmissible spongiform encephalopathy, traumatic brain injury any toxic encephalopahty, wobbly hedgehog syndrome and vasculitis.

The terms “brain damage” and “brain injury” (BI) as used herein are used interchangeably herein and refer to a pathological state of a subject characterized by the typically acute destruction or degeneration of brain cells, typically with an implication that the loss is significant in terms of functioning or conscious experience. These terms encompass a vast range of specific diagnoses. Brain injuries occur due to a wide range of internal and external factors. Preferably, said BI is an acquired brain injury. Another preferred type of BI is diffuse axonal injury (DAI).

The term “acquired brain injury” (ABI) is used, to differentiate brain injuries occurring after birth from injury due to a disorder or congenital malady.

The term “DAI” as used herein refers to a brain injury which is the result of traumatic shearing forces that occur when the head is rapidly accelerated or decelerated, as may occur in auto accidents, falls, and assaults. It usually results from rotational forces or severe deceleration. Vehicle accidents are the most frequent cause of DAI; it can also occur as the result of child abuse such as in shaken baby syndrome.

In preferred aspects of the invention, said BI is a traumatic brain injury (TBI).

The term “traumatic brain injury” (TBI) as used herein, is also known as “intracranial injury”, refers to a type of brain injury which occurs when an external force traumatically injures the brain. The term TBI also includes diffuse neuronal damage in the context of blast TBI.

The term “high impact TBI” as used herein refers to a trauma defined based on mechanism of injury as described (RTA/firearm/assault) by Minino et al. (2006), Natl Vital Stat Rep, 54:1-124 Forces involving the head striking or being struck by something, termed contact or impact loading, are the cause of most focal injuries, and movement of the brain within the skull, termed noncontact or inertial loading, usually causes diffuse injuries. TBI due to impact sufficient to cause axonal damage is here defined as high impact injury. Low impact TBI refers to impact unlikely to cause relevant damage to the brain tissue.

The term “blood,” as used herein, means the blood derivatives plasma and serum.

The term “microdialysate” as used herein refers to product of microdialysis. Microdialysis is a technique to monitor the composition of the extracellular space in living tissue. A physiological solution is slowly pumped through a microdialysis probe. With time, this solution equilibrates with the extracellular fluid (ECF), making it possible to measure the concentration of the molecules of interest in the microdialysate. Preferably a large pore size (100 kDa cutoff) is used. Smaller pore sizes (e.g. the routinely used 20 kDa cutoff membranes) are less suited for recovery of large molecular size proteins. However, for a smaller pore size is preferred for recovery of smaller eNfH fragments. Hence, lower cut-off membranes are suitable for use in aspects of the present invention, such as membranes with cut off values of 5 kDA, 10 kDa, 15, kDa, 20, kDa, 40, kDa, 50 kDa, 70 kDa 80 kDa or 100 kDa.

The term “subject,” as used herein, means a human or non-human animal, including but not limited to mammals such as a dog, cat, horse, cow, pig, rabbit, guinea pig, sheep, goat, primate, rat, and mouse. Since the immunogenic regions of NfH are well conserved across higher vertebrate species, the biomarker according to the invention is expected to work on avian and reptilian subjects also. By similar reasoning, assays based on the detection of a protein fragments described above are also likely to work on all higher vertebrate species.

The term “antibody” as used herein, or “immunoglobulin”, refers to a molecule comprising two heavy chains linked together by disulfide bonds and two light chains, each light chain being linked to a respective heavy chain by disulfide bonds in a “Y” shaped configuration.

It should be understood that when the terms “antibody” or “antibodies” are used, this is intended to include intact antibodies, such as polyclonal antibodies or monoclonal antibodies (mAbs), as well as proteolytic fragments thereof such as the Fab or F(ab′)2 fragments. Further included within the scope of the invention are chimeric antibodies; human and humanized antibodies; recombinant and engineered antibodies, and fragments thereof. Furthermore, the DNA encoding the variable region of the antibody can be inserted into the DNA encoding other antibodies to produce chimeric antibodies. Single chain antibodies fall within the scope of the present invention. Methods of production of such single chain antibodies are known.

The term “epitope” is meant to refer to that portion of any molecule capable of being bound by an antibody or a fragment thereof which can also be recognized by that antibody. Epitopes or antigenic determinants usually consist of chemically active surface groupings of molecules such as amino acids or sugar side chains and have specific three-dimensional structural characteristics as well as specific charge characteristics.

By reference to an “antibody that specifically binds” to another molecule is meant an antibody that binds the other molecule, and displays no substantial binding to other naturally occurring proteins other than those sharing the same antigenic determinants as the other molecule.

Unless otherwise defined, all technical terms used herein have the same meaning as commonly understood by one of ordinary skill in the art to which this invention belongs.

### PREFERRED EMBODIMENTS

The invention is based on the finding that eNfH protein fragments are detectable in samples in patients suffering from a disease associated with loss of neurons or brain damage and predict mortality following neuronal degeneration better than known biomarkers, especially those that can be detected in body fluids. The highest levels of these proteins were found following high impact TBI. The presence of these breakdown products is completely unexpected, as it was unknown that the enzyme Enterokinase, which is responsible for the cleavage of NfH into eNfH protein fragments, is present in neurons. In a prospective, clinical in vivo study 10 traumatic brain injury patients with a median Glasgow Coma Score of 9 were included, providing 640 cortical extracellular fluid samples for longitudinal data analysis. Following high impact TBI, microdialysate eNfH protein fragment levels were significantly higher (6.18±2.94 ng/mL) and detectable for longer (>4 days) compared to traumatic brain injury caused by a lower impact, (0.84±1.77 ng/mL, <2 days). During the initial 16 hours following traumatic brain injury, strong correlations were found between eNfH protein fragment levels and physiological parameters (systemic blood pressure, anaerobic cerebral metabolism, excessive brain tissue oxygenation, elevated brain temperature). eNfH protein fragment levels were of prognostic value, predicting mortality with great accuracy. Importantly, these protein fragments are very stable, do not form aggregates under physiological conditions and have a good solubility in water. Therefore, no special procedures for rapid sample preservation are required. Furthermore, they can be recovered from biological samples with a good recovery rate.

### The Biomarker

The invention provides a biomarker for the detection of brain damage or a disease associated with loss of neurons comprising a protein fragment of the neurofilament heavy chain (NfH) protein in a biological sample, wherein said protein fragment is a polypeptide selected from the group consisting of:
a polypeptide having the amino acid sequence selected from the group consisting of SEQ ID NO:1, SEQ ID NO:2, SEQ ID NO:3, SEQ ID NO:4 and SEQ ID NO:5, more preferably of SEQ ID NO:2, SEQ ID NO:3, SEQ ID NO:4 and SEQ ID NO:5, most preferably the polypeptides composed of amino acids 476-1026 and 476-986 of SEQ ID NO 6;
a protein fragment of the NfH protein having the amino acid sequence that is at least 60% identical to SEQ ID NO:1, at least 60% identical to SEQ ID NO:2, at least 60% identical to SEQ ID NO:3, at least 60% identical to SEQ ID NO:4 or at least 60% identical to SEQ ID NO:5;
an Enterokinase cleavage product of the NfH protein; and
a polypeptide that is a encoded by a naturally occurring allelic variant of the nucleic acid coding for a protein having the amino acid sequence selected from the group consisting of SEQ ID NO:1, SEQ ID NO:2, SEQ ID NO:3, SEQ ID NO:4 and SEQ ID NO:5, more preferably of SEQ ID NO:2, SEQ ID NO:3, SEQ ID NO:4 and SEQ ID NO:5, most preferably the polypeptides composed of amino acids 476-1026 and 476-986 of SEQ ID NO 6;
a polypeptide composed of amino acids 476-852 of SEQ ID NO 6, a polypeptide composed of amino acids 835-1026 of SEQ ID NO 6, a polypeptide composed of amino acids 835-986 of SEQ ID NO 6, a polypeptide composed of amino acids 852-1026 of SEQ ID NO 6, a polypeptide composed of amino acids 1-835 of SEQ ID NO 6, a polypeptide composed of amino acids 1-852 of SEQ ID NO 6, a polypeptide composed of amino acids 1-986 of SEQ ID NO 6, and fragments of NfH protein that are at least 60% identical to the indicated polypeptides and/or that are encoded by a naturally occurring allelic variant of the nucleic acid coding for NfH.

A preferred biomarker according to the invention is an eNfH polypeptide selected from the group consisting of a polypeptide with a calculated molecular weight of about 54 kDa having the amino acid sequence of SEQ ID NO:1, a polypeptide with a calculated molecular weight of about 39 kDa having the amino acid sequence of SEQ ID NO:2, a polypeptide with a calculated molecular weight of about 2 kDa having the amino acid sequence of SEQ ID NO:3, a polypeptide with a calculated molecular weight of about 15 kDa having the amino acid sequence of SEQ ID NO:4, a polypeptide with a calculated molecular weight of about 4 kDa having the amino acid sequence of SEQ ID NO:5, a polypeptide with a calculated molecular weight of about 60 kDa having the amino acid sequence of SEQ ID NO:14, a polypeptide with a calculated molecular weight of about 56 kDa composed of the amino acid sequence of SEQ ID NO 15, a polypeptide with a calculated molecular weight of about 41 kDa composed of amino acids 476-852 of SEQ ID NO 6, a polypeptide of about 21 kDa composed of amino acids 835-1026 of SEQ ID NO 6, a polypeptide of about 17 kDa composed of amino acids 835-986 of SEQ ID NO 6, a polypeptide of about 19 kDa composed of amino acids 852-1026 of SEQ ID NO 6, a polypeptide of about 92 kDa composed of amino acids 1-835 of SEQ ID NO 6, a polypeptide of about 94 kDa composed of amino acids 1-852 of SEQ ID NO 6, a polypeptide of about 109 kDa composed of amino acids 1-986 of SEQ ID NO 6, a polypeptide that is at least 60% identical to an indicated polypeptide and/or that is encoded by a naturally occurring allelic variant of the nucleic acid coding for NfH. It will be clear to the skilled person that the molecular weight of the polypeptide as determined by migration in gel-electrophoresis may differ from the calculated molecular weight, for example due to the presence of post-translational modifications such as phosphorylation and/or glycosylation.

Highly preferred is an enterokinase cleavage product of the NfH protein of SEQ ID NO:6 comprising a polypeptide other than the fragment of amino acids 1-476.

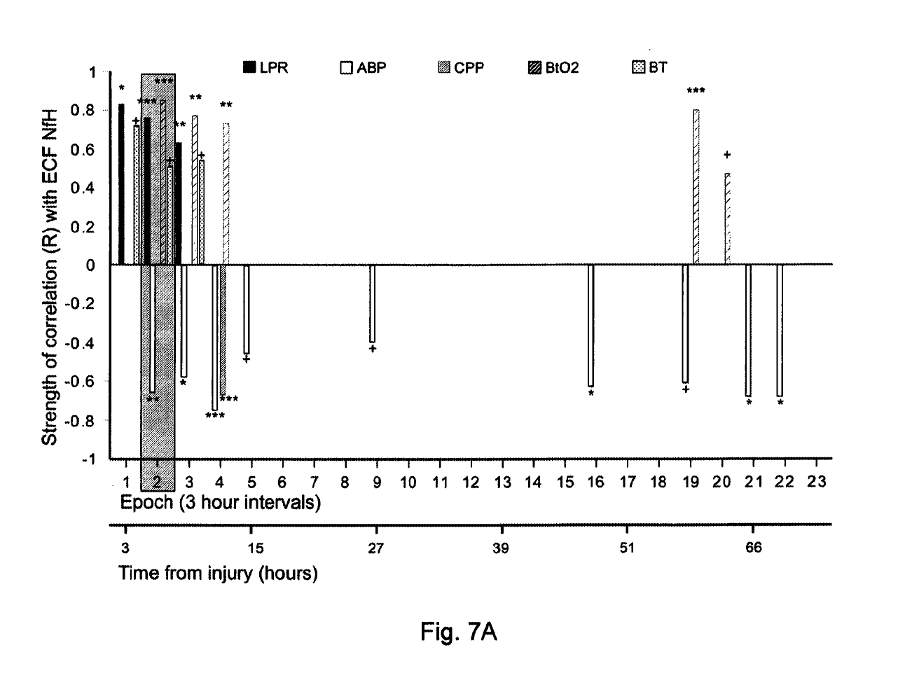

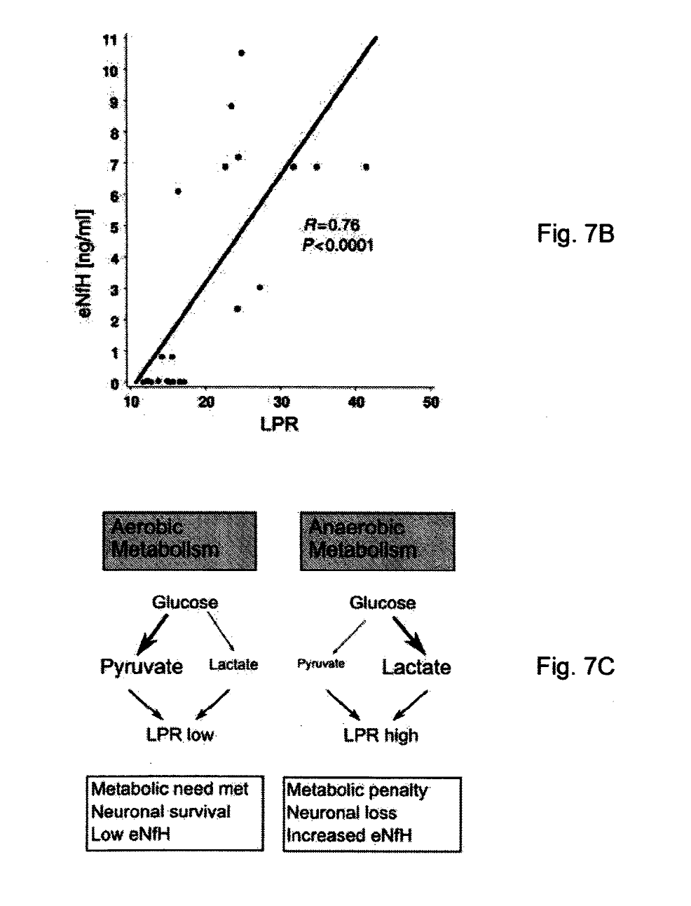

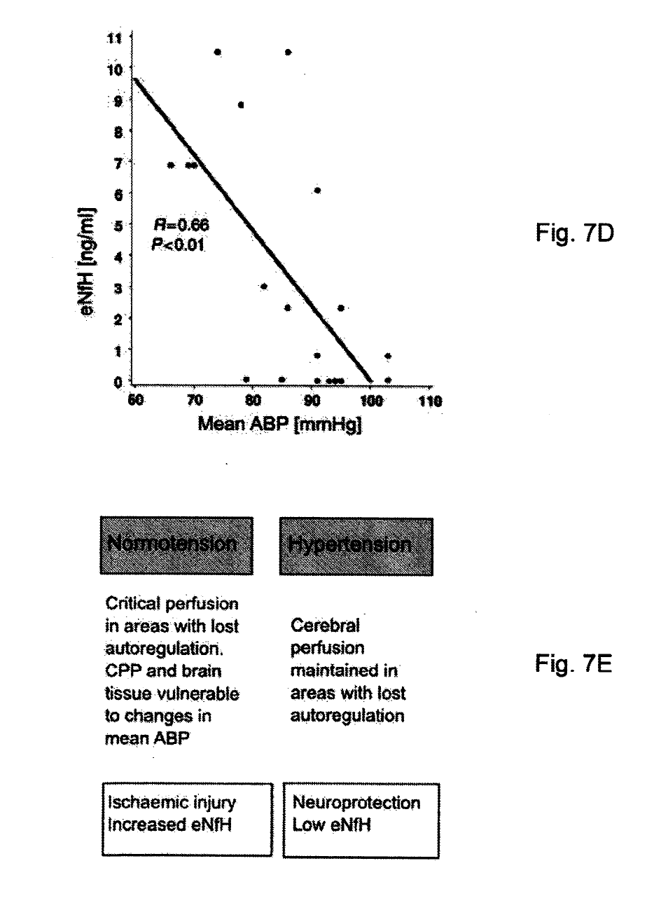

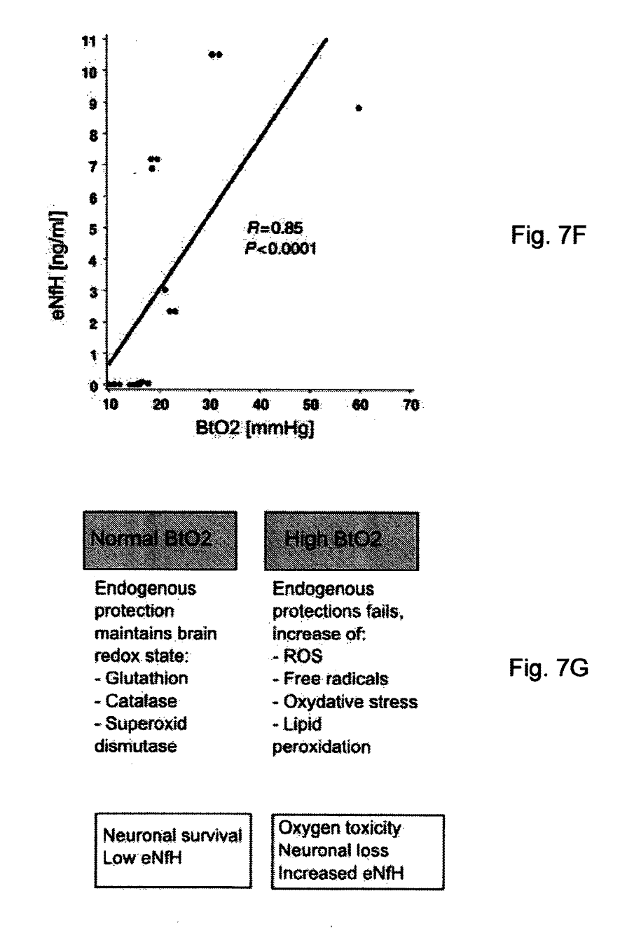

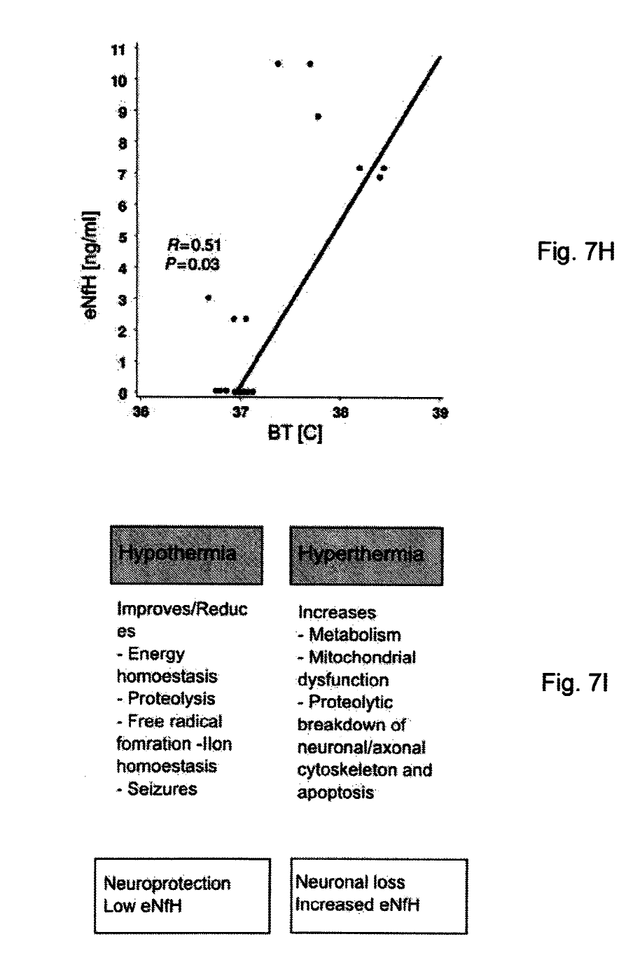

The biomarker was identified based on the observation that levels of the eNfH protein fragments were correlated with physiological parameters related to severity of injury and outcome in TBI, including lactate to pyruvate ratio (LPR), arterial blood pressure (ABP), brain tissue oxygenation (BtO2) and brain temperature (BT). The four parameters investigated are summarised in FIG. 7A-D. The following observations were made that further support the relevance of this biomarker for the detection of brain damage or a disease associated with loss of neurons:

1. Strong correlations were found between levels of eNfH protein fragments and the cortical extracellular fluid lactate to pyruvate ratio. These correlations indicate neuronal loss (FIG. 7A). It is therefore contemplated that the eNfH protein fragment levels can replace the lactate to pyruvate ratio as a biomarker for neuronal loss.

2. Correlations were found between levels of eNfH protein fragments and the systemic blood pressure. It is therefore contemplated that levels eNfH protein fragments may be measured to monitor arterial blood pressure resuscitation (FIG. 7B).

3. Moderately strong correlations were found between levels of eNfH protein fragments and BtO₂ levels. This suggests that increasing BtO₂ may saturate systems of endogenous protection. The resulting O₂ toxicity may cause neuronal injury resulting in increased levels of eNfH. It is contemplated that the level of an eNfH protein fragment is used as a biomarker for monitoring a treatment comprising normobaric hyperoxia as a neuroprotective treatment (FIG. 7C).

4. Levels of eNfH protein fragments were related to the brain temperature. This suggests that higher brain temperature may accelerate mechanisms leading to neuronal apoptosis resulting in an increase of eNfH polypeptide levels. Importantly, correlations between brain temperature and eNfH levels were most significant during the acute phase during which rapid hypothermia might have its strongest neuroprotective mechanism. It is contemplated that the presence of elevated eNfH levels may be used as an exclusion criterium and/or secondary outcome measure for treatments comprising hypothermia in the treatment brain damage, preferably TBI (FIG. 7D).

It is shown herein that the level of an eNfH protein fragment in the initial 12 hours following TBI was predictive for mortality with an odds ratio of 7.68. The odds ratio for levels of a protein fragment as described above compared favourable to the Glasgow Coma Score (GCS, odds ratio 4.13) and other clinical predictors derived from the large IMPACT dataset on 8509 TBI patients (Motor score of the GCS 1.0-5.7 (Steyerberg et al., 2008)). Quantification of these proteins allows in vivo monitoring of neurodegeneration in TBI. The data also indicate that levels of these protein fragments predict poor functional outcome.

In conclusion, the protein fragments as described above are specific biomarkers for detecting the loss of neurons and may inter alia be detected in the cortical extracellular fluid.

Preferably, said increased amount is at least a measurable amount, preferably 0.03, 0.05, 0.1, 0.2, 0.3, 0.4, 0.5, 0.6, 0.7, 0.8, 0.84, 0.9, 1, 2, 3, 4, 5, 6, to 6.18 ng/ml. Preferably, the increase is 1.5, 2, 2.5, 3, 4, 5, 6 or 7 fold compared to a reference value from a healthy individual or an individual not suffering from high impact TBI. This is based on the finding that these amounts are highly indicative of neuronal damage. In patient suffering from high impact TBI, the averaged eNfH levels over the initial 24 hours were 7-fold higher (6.18±2.94 ng/mL) following high impact TBI compared to TBI caused by a lower impact (0.84±1.77 ng/mL). Moreover, eNfH levels remained for longer on a high level following high impact TBI (data not shown). In patients suffering from traumatic brain injury after a fall, eNfH protein fragment levels declined steadily to become non-detectable after an average of two days. This indicates that the level of the protein fragment as described herein can be used to estimate the level of neuronal loss.

It is preferred that the protein fragment as described above has an equal folding as its natural form. It is also preferable that the protein fragment as described above has the same post translational modifications as its natural form. Otherwise, the molecular weight is different. Post translational modification also may influence the immunoreactivity of a protein. The natural form of NfH is extensively phosphorylated. It is therefore preferred that the protein fragment as described above is also phosphorylated. The C-terminal tail domain is typically phosphorylated at serine and threonine residues present in the KSP (Lys-Ser-Pro) repeats. Also the N-terminal domain of the NfH protein is typically phosphorylated. Therefore, the eNfH protein fragment which contains the N terminal tail of the NfH protein (eNfH 1-476) is preferably also phosphorylated.

It is further preferred that the KSP repeats present in the eNfH 476-1026 and eNfH 835-1026 protein fragments comprise an O-GlcNAc modification.

Preferably, said protein fragment is an Enterokinase cleavage product of the NfH protein. Preferably, said protein fragment is an Enterokinase cleavage product having a molecular weight selected from the group consisting of 101-112 kDa or 94-104 kDa, 89-99 kDa, 35-39 kDa and 25-28 kDa, more preferably of 101-112 kDa and 94-104 kDa, as determined by migration in gel-electrophoresis. Said NfH protein may be of any species, but is preferably of a vertebrate animal, more preferably of a mammalian animal. Preferably it is from dog, mouse, rat, guinee pig, chicken, chimpanzee, bovine, but most preferably from a human animal.

Enterokinase cleavage products of products encoded by orthologs of the NfH gene are therefore also part of the invention. Sequence similarities of some orthologs of the human NfH protein and the coding gene in dog (85.5% nucleotide, 90.67% amino acid), chimpanzee (99.38% nucleotide, 99.2% amino acid), cow (85.89% nucleotide, 88.82% amino acid), rat (78.48% nucleotide, 79.16% amino acid), mouse (80.97% nucleotide, 81.67% amino acid) and chicken (65.15% nucleotide, 64.86% amino acid) are quite high. Preferably, the similarity in amino acid sequence with SEQ ID NO:1 is at least 79, 81, 88, 90, 91, 92, 93, 94, 95, 96, 97, 98 or 99%. Preferably, the similarity in amino acid sequence with SEQ ID NO:2 is at least 79, 81, 88, 90, 91, 92, 93, 94, 95, 96, 97, 98 or 99%. Preferably, the similarity in amino acid sequence with SEQ ID NO:3 is at least 79, 81, 88, 90, 91, 92, 93, 94, 95, 96, 97, 98 or 99%. Preferably, the similarity in amino acid sequence with SEQ ID NO:4 is at least 79, 81, 88, 90, 91, 92, 93, 94, 95, 96, 97, 98 or 99%. Preferably, the similarity in amino acid sequence with SEQ ID NO:5 is at least 79, 81, 88, 90, 91, 92, 93, 94, 95, 96, 97, 98 or 99%. Preferably, the similarity in amino acid sequence with SEQ ID NO:14 is at least 79, 81, 88, 90, 91, 92, 93, 94, 95, 96, 97, 98 or 99%. Preferably, the similarity in amino acid sequence with SEQ ID NO:15 is at least 79, 81, 88, 90, 91, 92, 93, 94, 95, 96, 97, 98 or 99%.

A further preferred biomarker has similarity in amino acid sequence of at least 79, 81, 88, 90, 91, 92, 93, 94, 95, 96, 97, 98 or 99% with a polypeptide selected from the group consisting of a polypeptide with a calculated molecular weight of about 54 kDa having the amino acid sequence of SEQ ID NO:1, a polypeptide with a calculated molecular weight of about 39 kDa having the amino acid sequence of SEQ ID NO:2, a polypeptide with a calculated molecular weight of about 2 kDa having the amino acid sequence of SEQ ID NO:3, a polypeptide with a calculated molecular weight of about 15 kDa having the amino acid sequence of SEQ ID NO:4, a polypeptide with a calculated molecular weight of about 4 kDa having the amino acid sequence of SEQ ID NO:5, a polypeptide with a calculated molecular weight of about 60 kDa having the amino acid sequence of SEQ ID NO:14, a polypeptide with a calculated molecular weight of about 56 kDa composed of the amino acid sequence of SEQ ID NO 15, a polypeptide with a calculated molecular weight of about 41 kDa composed of amino acids 476-852 of SEQ ID NO 6, a polypeptide of about 21 kDa composed of amino acids 835-1026 of SEQ ID NO 6, a polypeptide of about 17 kDa composed of amino acids 835-986 of SEQ ID NO 6, a polypeptide of about 19 kDa composed of amino acids 852-1026 of SEQ ID NO 6, a polypeptide of about 92 kDa composed of amino acids 1-835 of SEQ ID NO 6, a polypeptide of about 94 kDa composed of amino acids 1-852 of SEQ ID NO 6, and/or a polypeptide of about 109 kDa composed of amino acids 1-986 of SEQ ID NO 6.

Natural allelic variants of the NfH gene naturally occur and give occasionally rise to allelic variants of the NfH protein. A skilled person can easily recognize such allelic variants as such, for instance, based on its function, the typical phosphorylation and/or presence in neurons. As it is also known which at which sites Enterokinase cleaves, the amino acid sequences of the resulting protein fragments can be determined. The Enterokinase cleavage products of these natural variants of NfH are part of the invention.

### Methods for Diagnosis and Prognosis of Neuronal Loss

The invention further provides a method for diagnosis and/or prognosis of brain damage or a disease associated with a loss of neurons in a subject, comprising steps of providing a biological sample of said subject; detecting in said biological sample a biomarker according to the invention, comparing the amount of said biomarker in said biological sample with reference values, and determining whether loss of neurons has occurred or determining the level of loss of neurons based on the comparison. In a preferred embodiment, an elevated level of said biomarker is indicative of the presence of said brain damage or a disease associated with a loss of neurons in the case of diagnosis or the progression of said brain damage or a disease associated with a loss of neurons in the case of prognosis. The invention further provides a method for diagnosis and/or prognosis of brain damage or a disease associated with a loss of neurons in a subject, comprising steps of detecting a biomarker according to the invention in a biological sample from the subject, comparing the amount of said biomarker in said biological sample with reference values, and determining whether loss of neurons has occurred or determining the level of loss of neurons based on the comparison. In a preferred embodiment, an elevated level of said biomarker is indicative of the presence of said brain damage or a disease associated with a loss of neurons in the case of diagnosis or the progression of said brain damage or a disease associated with a loss of neurons in the case of prognosis. It will be clear to a skilled person that one or more of the biomarkers may be combined in the methods of the invention. The level of biomarkers in healthy individuals can be measured and recorded to provide reference values of the levels of biomarkers. Specifically, the recorded level of biomarker in healthy individuals forms a base line value that can be compared with those levels in patients that are suspected of suffering from a disease associated with loss of neurons or brain damage. Because the level of biomarkers in an individual may vary significantly during his or her life span, different base line values can be established for different age and gender groups.

Preferably, said reference values are values for healthy subjects in the case of diagnosis, or values of the same subject at earlier points in time in the case of prognosis.

Said method may comprise determining the level of said protein fragment as described above once or several times, e.g., several times within a determined period of time. For example, a diagnostic method may comprise obtaining a first biological sample of a subject, and obtaining a second biological sample several hours or days (e.g., 1, 2, 3, or 7 days) or weeks (e.g., 1, 2, 3 or 4 weeks) or months (e.g., 1, 2, 3, 6, 10 or more months) or years later. A change in the level of protein fragment of an eNfH protein fragment within the two samples may be indicative that a disease, e.g., a neurodegenerative disease, is evolving in the subject. An increase in the level of an eNfH protein fragment with time in a subject may indicate that the subject is developing a disease. A decrease in the level of protein of an eNfH protein fragment with time in a subject may indicate that the disease or at least one or more symptoms thereof is being treated or prevented effectively.

A diagnosis and/or prognosis of a disease may also comprise monitoring another characteristic of the disease, e.g., the presence or absence or level of a biomarker of the disease. For example, the diagnosis of Alzheimer's disease as described herein may be combined with the detection of β-amyloid plaques. Preferably, the method also comprises determining a further biomarker for diagnosis and/or prognosis of a disease as defined herein or brain damage, including GCS, a parameter based on pyruvate and/or lactate levels, preferably the pyruvate-to-lactate ratio and/or a physiological parameter, including the systemic blood pressure, the level of anaerobic cerebral metabolism, the level of (excessive) brain tissue oxygenation and (elevated) brain temperature.

The diagnosis and/or prognosis of a subject as having or being likely to develop a disease, e.g., a neurodegenerative disease, may be followed by the treatment of the subject. In an illustrative embodiment, a method comprises first determining whether a subject has a disease associated with the loss of neurons or is likely to develop a disease associated with the loss of neurons and second administering to a subject who was diagnosed as having or likely to develop the disease a therapeutically effective amount of an agent for treating the disease associated with the loss of neurons.

In another embodiment, a subject is treated, and the efficacy of the treatment or the progression of the disease is determined. A treatment as referred to herein above, may be any treatment known in the art. The efficacy of the treatment or the progression of the disease may be determined by measuring the level of the protein fragment as described above in the subject being treated. Measurements may be conducted on a regular basis, e.g., every day, every other day, once a week, once a month or once a year.

The step of providing a biological sample derived from the subject can be performed by conventional medical techniques. A biological sample can be from any site in the body of the subject. While eNfH fragments are expected to accumulate in the cerebrospinal fluid following neuronal injury, and could be assayed there, it is expected that eNfH protein fragments can also be detected in other types of biological samples. A preferred biological sample is a sample from a body fluid, because body fluids are easily available. Preferred body fluids are blood, plasma and cerebrospinal fluid (CSF). In alternative embodiments, the biological sample is a brain tissue sample, preferably a cortical brain tissue. Preferably, said biological sample is a microdialysate of the extracellular fluid of the brain, preferably obtained using a microdialysis membrane having a cut-off of around 100 kDa. The extracellular fluid (ECF) or interstitial fluid of the brain is defined as the fluid in between the brain cells. The ECF or interstitial fluid is understood to diffuse into the CSF. Specific proteins released from dying neurons, such as eNFH are released into the ECF from where they equilibrate with the CSF and the blood.

A highly preferred biological sample is a blood sample. Blood is much more easily obtained than CSF.

Preferably, said biological sample is a pre-delivered sample collected within a week, preferably within 12 hours after the onset of said brain damage or a disease associated with a loss of neurons. An advantage thereof is that these samples result in more accurate prognosis or diagnosis.

Suitable subjects for use in the invention can be any animal species expressing NfH in neurons. The subject can therefore be any living or dead mammal such as dog, cat, horse, cow, pig, rabbit, guinea pig, sheep, goat, primate, rat, or mouse. It is expected that this assay will work at least on avian and reptilian species, if not also amphibian, fish and cell culture models. A preferred subject for the methods of the invention is a human being. Particularly preferred are subjects suspected of having or at risk for developing traumatic or non-traumatic neuronal injuries, such as victims of neuronal injury caused by traumatic insults (e.g., gunshot wounds, automobile accidents, sports accidents), ischemic events (e.g., stroke, cerebral hemorrhage, cardiac arrest) and neurodegenerative disorders.

Preferably, a subject in aspects of the invention is a patient suffering from TBI. Preferably, the TBI patient does not suffer from (i) polytrauma, (ii) significant thoracic or abdominal injuries, and/or (iii) documented pre-admission hypotensive (systolic blood pressure<90 mmHg) and/or hypoxic (peripheral oximetry saturation<90%) episodes.

Preferably, a method for prognosis comprises steps of prognosticating a clinical endpoint, preferably including:

(i) survival on discharge from hospital and/or

(ii) the success of treatment, preferably expressed on an objectivised scale.

Preferably said scale is the Glasgow Outcome Score (GOS) at a certain time point after injury, preferably 3 months and/or 6 months. Preferably said GOS is divided into two classifications consisting of unfavourable recovery (GOS: 1 indicates death; 2, persistent vegetative state; and 3, severe disability) and favourable recovery (GOS: 4 indicates moderate disability; 5, good recovery). The GOS criteria are preferably as described in Teasdale et al., 1998, J Neurotrauma, 15:587-597.

For detecting said protein fragment, any method which is suitable for detecting specific proteins and polypeptides may be used. Numerous suitable techniques are known for analyzing the presence or detecting the level of proteins or polypeptides. Preferably, said protein fragment is detected using a detection assay based on Mass Spectrometry techniques, separation techniques (based on molecular size and/or electro negativity), preferably HPLC, or immunological techniques or a combination of these techniques. Said immunological techniques include the use of antibodies that specifically bind to the protein fragment in immunoassays. Preferred immunoassays include immunoblotting (e.g., Western blotting), ELISA, electrochemiluminescence (ECL) based assays, PCR and quantitative PCR techniques including tagging of antibodies, radioimmunoassay (RIA), immunofluorescence, immunohistochemical staining and analysis, and similar techniques.

In preferred embodiments of said method, the step of detecting the biomarker is performed by using Mass spectrometry (MS), preferably by tandem mass spectrometry (MS-MS) or by Matrix-assisted laser desorption/ionization (MALDI). Mass spectrometry provides a powerful means of determining the structure and identity of complex organic molecules, including proteins and peptides. A sample compound is bombarded with high-energy electrons causing it to fragment in a characteristic manner. The fragments, which are of varying weight and charge, are then passed through a magnetic field and separated according to their mass/charge ratios. The resulting characteristic fragmentation signature pattern of the sample compound is used to identify and quantitate that compound. A typical MS procedure comprises the following steps:

1. a sample is loaded onto the MS instrument, by applying the sample optionally (in case of a special form of MS called MALDI) together with a matrix on a mass spectrometric sample support and drying the sample or the mixture on the support by evaporation of the solvents.

2. the components of the sample are ionized by one of a variety of methods (e.g., by impacting them with an electron beam), which results in the formation of charged particles (ions)

3. the positive ions are then accelerated by an electric field

4. computation of the mass-to-charge ratio (m/z) of the particles based on the details of motion of the ions as they transit through electromagnetic fields, and

5. detection of the ions, which in step 4 were sorted according to m/z.

The technique can be used for identifying compounds in the sample as a protein fragment of eNfH as described herein, by determining the structure of the protein fragment from its fragmentation pattern.

An advantage of the MALD technique is that it is a soft ionization technique used in mass spectrometry, which is specifically suitable for the analysis of the protein fragment as described above, which tend to be fragile and fragment when ionized by more conventional ionization methods. The matrix as described above is used to protect the protein fragment as described above from being destroyed by direct laser beam and to facilitate vaporization and ionization.

The matrix consists of crystallized molecules, of which the three most preferably used are 3,5-dimethoxy-4-hydroxycinnamic acid (sinapinic acid), α-cyano-4-hydroxycinnamic acid (alpha-cyano or alpha-matrix) and 2,5-dihydroxybenzoic acid (DHB). The matrix solution is mixed with the protein fragment-sample. The organic solvent allows hydrophobic molecules to dissolve into the solution, while the water allows for water-soluble (hydrophilic) molecules to do the same. This solution is spotted onto a MALDI plate (usually a metal plate designed for this purpose). The solvents evaporate, leaving only the recrystallized matrix, together with the protein fragments dispersed throughout the crystals. The matrix and the protein fragments are thus co-crystallized in a MALDI spot.

Other MS applications include MALDI-TOF MS mass spectrometry, MALDI-FT mass spectrometry, MALDI-FT-ICR mass spectrometry, MALDI Triple-quad mass spectrometry.

An alternative embodiment in aspects of the invention a protein microarray for simultaneous binding and quantification of the protein fragments as described above is used. The protein microarray may suitably consist of the antibody as described herein bound to a defined spot position on a support material. The array is exposed to a complex protein sample. The antibody is able to bind the protein fragment as described herein from the biological sample. The binding of the specific protein fragment to the individual spots can then be monitored by quantifying the signal generated by each spot. Protein microarrays may include two-dimensional microarrays constructed on a planar surface, and three-dimensional microarrays which use a Flow-through support.

Types of protein microarray set-ups include reverse phase arrays (RPAs) and forward phase arrays (FPAs). In RPAs a small amount of a tissue or cell sample is immobilized on each array spot, such that an array is composed of different patient samples or cellular lysates. In the RPA format, each array is incubated with one detection protein (e.g., antibody), and a single analyte endpoint is measured and directly compared across multiple samples. In FPAs, capture agents, usually antibodies or antigens, are immobilized onto the surface and act as a capture molecule. Each spot contains one type of immobilized antibody or capture protein. Each array is incubated with one test sample, and multiple analytes are measured at once.

One of the most common forms of FPAs is an antibody microarray. Antibody microarrays can be produced in two forms, either by a sandwich assay or by direct labelling approach. The sandwich assay approach utilizes two different antibodies that recognize two different epitopes on the target protein. One antibody is immobilized on a solid support and captures its target molecule from the biological sample. Using the appropriate detection system, the labelled second antibody detects the bound targets. The main advantage of the sandwich assay is its high specificity and sensitivity. High sensitivity is achieved by a strong reduction of background, yielding a high signal-to noise ratio. In addition, only minimal amounts of labelled detection antibodies are applied in comparison to the direct labelling approach were a large amount of labelled proteins are present in a sample. The sandwich immunoassay format can also be easily applied to the field of microarray technology, and such immunoassays can be applied to the protein microarray format to quantify the biomarkers as described herein in conditioned media and/or patient sera.

In the direct labelling approach, all proteins and protein fragments in a sample are labelled with a fluorophore. Labelled proteins and protein fragments that bind to the protein microarray such as to an antibody microarray are then directly detected by fluorescence. Also, proteins from two different biological samples may be labelled with different fluorophores. These two labelled samples are then mixed together in an unknown ratio and applied to an antibody microarray. This approach allows (direct) comparisons between diseased and healthy, or treated and untreated samples. Direct labelling has the advantage that the direct labelling method only requires one specific antibody is required to perform an assay.

Miniaturized and multiplexed immunoassays may also used to screen a biological sample for the presence or absence of proteins.

In a preferred embodiment of aspects the invention, the detection or capture agents, preferably antibodies are immobilized on a solid support, preferably on a polystyrene surface. In another most preferred embodiment, the detection or capture agents are spotted or immobilized in duplicate, triplicate or quadruplicate onto the bottom of a well of a microplate.

In a further preferred embodiment, electrochemiluminescence (ECL) immunoassay technologies are used for the detection of eNfH cleavage products. An ECL-based immunoassay applies a similar format as conventional ELISA assays, i.e. two different antibodies capturing an antigen (sandwich).

### Antibodies

Further provided is an antibody or a molecule having the antigen-binding portion of an antibody which specifically detects a protein fragment as described above. The antibody may be a polyclonal or monoclonal antibody as well as an antibody fragment or a portion of a immunoglobulin molecule that can specifically bind the same antigen as the intact antibody molecule. In a preferred embodiment, said antibody is directed to the C- or N-terminal region of the protein fragment as described above, more preferably the terminal regions which flank the EK cleavage site. Preferably, said antibody that specifically binds does not bind to a full length NfH protein. Preferably, said antibody does not bind to fragments of the NfH protein which are not a cleavage product of Enterokinase. Preferably, said antibody is directed to an epitope which comprises the sequences recognized by Enterokinase. Preferably, said epitope comprises the amino acids E-E-E-E-K, E-E-E-K or, E-D-D-K.

Preferably, said antibody is a monoclonal antibody. MAbs may be obtained by methods known to those skilled in the art. The mAbs of the present invention may be of any immunoglobulin class including IgG, IgM, IgE, IgA, and any subclass thereof. Methods for producing mAbs are well known in the art and include the use of hybridomas. A hybridoma producing a mAb may be cultivated in vitro or in vivo. High titers of mAbs can be obtained in in vivo production where cells from the individual hybridomas are injected intraperitoneally into mice, preferably pristine-primed Balb/c mice, to produce ascites fluid containing high concentrations of the desired mAbs. MAbs of isotype IgM or IgG may be purified from such ascites fluids, or from culture supernatants, using column chromatography methods well known to those of skill in the art.

Besides the conventional method of raising antibodies in vivo, antibodies can be generated in vitro using phage display technology. Such in vitro methods for the production of recombinant antibodies are much faster compared to conventional antibody production and recombinant antibodies can be generated against an enormous number of antigens.

To generate recombinant monoclonal antibodies one can use various methods based on phage display libraries to generate a large pool of antibodies with different antigen recognition sites. Such a library can be made in several ways: One can generate a synthetic repertoire by cloning synthetic CDR3 regions in a pool of heavy chain germline genes and thus generating a large antibody repertoire, from which recombinant antibody fragments with various specificities can be selected. One can use the lymphocyte pool of humans as starting material for the construction of an antibody library. It is possible to construct naive repertoires of human IgM antibodies and thus create a human library of large diversity. Protocols for bacteriophage library construction and selection of recombinant antibodies are well known.

### Kit-of-Parts

The invention also provides a kit-of-parts comprising a protein fragment as described above and/or an antibody according to the invention. In a preferred embodiment, said antibody is immobilized to a solid support, preferably on a polystyrene surface. The kit preferably also includes a labelled antibody specific for detecting said antibody according to the invention. In another preferred embodiment, the kit further comprises a second antibody according to the invention which detects a second epitope on the same protein fragment as defined herein. This enables a “sandwich” detection of the protein fragment, which decreases non specific background signals. Preferably, the kit also includes instructions for using the kit to detect neuronal loss in a subject. Hence, the kit may optionally comprise a biomarker or detection antibody for the biomarker in combination with a written instruction for performing a method for diagnosis and/or prognosis of brain damage or a disease associated with a loss of neurons in a subject according to the present invention. The kit may typically include an eNfH fragment-specific polyclonal, monoclonal or recombinant antibody immobilized on ELISA plates, glass slides or other suitable substrates. The immobilized antibody is incubated with the biological sample allowing binding of the specific eNfH fragment that may be contained in the sample. The binding of the specific protein fragment may be detected by the labelled antibody. The presence of the detection antibody is visualized and quantified by detection agents such as enzyme-linked antibodies reactive with the detection antibody. The presence of the enzyme linked antibody is then detected using for instance chromogenic substrate molecules appropriate for the enzyme.

Quantitation of the signal can then be performed by optical density measurements at the wavelength optimum for the particular chromagen. More complex approaches that utilize surface plasmon resonance, fluorescence resonance energy transfer or other techniques which involve the use of specialized equipment to assay binding may have advantages in terms of quantifying binding and for high-throughput applications.

The protein fragments as described herein may be prepared by any methods for making proteins or can be isolated from natural sources. The protein fragment is in certain preferred embodiments a recombinant protein. It can be made by any known recombinant technique. Preferably, it is produced in eukaryotic cells in order to achieve correct folding and/or post translational modification.

The Enterokinase cleavage products are suitably prepared by a combining a NfH protein and Enterokinase in solution, allowing the cleavage of said NfH protein; and preferably isolating the resulting protein fragment. Enterokinases are commercially available (Roche, EC 3.4.21.9, Cat No 11 334 115 001). The resulting cleavage products may be used as positive controls in the indicated detection assays. The Enterokinase cleavage products may also be prepared by expressing one or more of the polypeptides in vitro using a cell-free protein synthesis system and/or by expressing one or more of the polypeptides in expression systems such as insect cells, bacteria, yeast and/or mammalian cells. For this, expression cassettes may be generated that comprise a relevant part of the genomic DNA or cDNA encoding one or more of the polypeptides and appropriate control elements that mediate expression of the genomic DNA or cDNA in a selected expression system. It will be clear to the skilled person that translation start and stop signals might have to be added to a relevant part of the genomic DNA or cDNA encoding one or more of the polypeptides to ensure expression of the polypeptides. A preferred expression system is a system that is capable of posttranslational modification (e.g. phosphorylation and/or glycosylation) of the expressed polypeptide such that it mimics the Enterokinase cleavage product that is identified in the brain after trauma. A preferred expression system is a mammalian cell, preferably a human cell, more preferred a human neuronal cell.

A preferred kit according to the invention is an ELISA kit or an electrochemiluminescence (ECL)-based immunoassay for the detection of eNfH cleavage products. Electrochemiluminescence (ECL) detection uses labels that emit light when electrochemically stimulated. An ECL-based immunoassay is very suitable for assaying low abundance proteins due to a superior signal to noise ratio. It applies a similar format as conventional ELISA assays, i.e. two different antibodies capturing the biomarker antigen of the present invention in ‘sandwich’ format, using an ECL label. An ECL label is a chemical substance that, when electrochemically oxidized or reduced under appropriate conditions, emits light. The term “ECL label” or “electrochemiluminescent label” refers to the substance itself, to a chemical derivative that has been modified to allow attachment to substrate or other reagent, or to a chemical derivative that is attached to a substrate or other reagent. The term “ECL label” also refers to the various products and/or intermediates formed from the label during the ECL-generating reaction. Numerous ECL labels have been reported in the literature (see the review by Knight et al., Analyst, 119, 879, 1994). Useful ECL labels include polyaromatic hydrocarbons (e.g., 9,10-diphenylanthracene, rubrene, phenanthrene, pyrene, and sulfonated derivatives thereon, organometallic complexes (e.g., complexes containing lanthanides, ruthenium, osmium, rhenium, platinum, chromium, and/or palladium), organic laser dyes, quantum dots and coded nanoparticle tags, and chemiluminescent species (e.g., diacyl hydrazides such as luminol, acridinium esters, luiferase, and lucigenin). The ECL signal can be advantageously increased by using labels comprising a polymer or particle platform linked to a plurality of individual ECL labels (see, e.g., U.S. Pat. No. 5,679,519). Advantageous ECL labels are luminol and polypyridyl (especially bipyridyl or phenanthrolyl)-containing complexes of ruthenium, osmium or rhenium (see, e.g., the complexes described in U.S. Pat. No. 5,714,089, U.S. Pat. No. 5,591,581, U.S. Pat. No. 5,597,910, and published PCT application WO87/06706. The most advantageous ECL labels are ruthenium tris-bipyridyl (RuBpy) and its derivatives including tris(2,2′-bipyridyl)ruthenium(II) [Ru(bpy)₃²⁺]

Although methods and materials similar or equivalent to those described herein can be used in the practice or testing of the present invention, suitable methods and materials are described below.

### Examples

### Patients

Patients suffering from a traumatic brain injury who were admitted to the Neurosurgical Intensive Care Unit at the National Hospital for Neurology and Neurosurgery were recruited between January 2006 and February 2007. Inclusion criteria were (i) traumatic brain injury; (ii) age≧16 years; (iii) requirement for intra-cranial pressure monitoring in accordance with published guidelines [(PMID (PubMEd): 10937893) 2000] and (iv) that written, informed, witnessed consent could be obtained from the next of kin. Exclusion criteria included (i) polytrauma; (ii) significant thoracic or abdominal injuries; (iii) documented pre-admission hypotensive (systolic blood pressure<90 mmHg) or (iv) hypoxic (peripheral oximetry saturation<90%) episodes. High-velocity impact traumatic brain injury was defined based on mechanism of injury (road traffic accidents, fire-arm, and assault) (Kirkpatrick J B (1983) Acta Neurochir Suppl 32:115-7; Walilko et al. (2005) Br J Sports Med 39:710-9; Minino et al. (2006) Natl Vital Stat Rep, 54:1-124, Langlois et al. (2006) J Head Trauma Rehabil, 21:375-378 and Adekoya & Majumder (2004) Public Health Rep, 119:486-492). Severity of injury was additionally classified clinically based on the admission Glasgow Coma Score (GCS) into mild (GCS 13-15), moderate (GCS 9-12) and severe (GCS≦8).

### Brain Imaging

All patients underwent computerized tomography (CT). The CT brain imaging was classified according to the Marshall criteria (Marshall et al. (1992) J Neurotrauma 9:S287-92; Steyerberg et al. (2008) PLoS Med 5:e165. discussion e165), the presence of traumatic subarachnoid blood (Steyerberg et al. (2008) supra) or an extradural haematoma (Steyerberg et al. (2008) supra).

### Procedures and Sample Collection

The microdialysis catheter (100 kDa molecular weight cut-off, CMA71, 20 mm length, 0.6 mm diameter polyamide membrane) was implanted through a triple lumen intracranial bolt (Technicam Ltd, Newton, Abbott, UK) in pericontusional tissue, in cases of contusion injury, or into the right frontal cortex, in cases of diffuse axonal injury, in agreement with consensus guidelines (Bellander et al. (2004) Intensive Care Med 30:2166-9). The catheter was perfused with commercially available artificial CSF (NaCl 147 mM, KCl 2.7 mM, CaCl₂ 1.2 mM, MgCl₂ 0.85 mM as supplied by CMA). The catheter was perfused at a constant flow rate of 0.3 μL/min. The system was allowed to equilibrate for one hour prior to starting sample collection as per consensus guideline (Bellander et al. (2004) supra). Samples were collected on an hourly basis and frozen within 10 minutes of collection. The position of the gold labelled probes was verified on subsequent computerised tomography (CT) scans (except in Patient 5 were the catheter was inserted very late and Patient 9 were the catheter was removed very early). Intracranial pressure (ICP) (Codman microsensor, Randolph, Ma, USA), brain temperature (BT) and calibrated brain tissue oxygenation (BtO2) were recorded continuously (1 min intervals) using the LICOX device (Integra LifeSciences Corporation). Hourly mean values for these variables were calculated and confirmed by one physician.

### Cortical Brain Tissue Samples

Post-mortem (PM) cortical tissue from TBI cases (n=4) were taken from areas close to macroscopically visible contusions. Control cortical PM tissue was taken from two patients who died of a non-neurological condition. In addition, control brain tissue samples were collected from patients undergoing surgery for intractable epilepsy (n=3). Tissue samples were fixed with formalin prior to embedding in paraffin.

### Patient Management

Patients were sedated with propofol and fentanyl and received protocol-guided stepwise therapy based on the Brain Trauma Foundation guidance (Kitson & Desai 2005 Critical Care 9:E28) to maintain a cerebral perfusion pressure (CPP) above 60 mmHg. As part of the ICP management patient were mechanically ventilated to maintain a PaCO₂ of 4.0 to 4.5 kPa. Blood glucose was maintained between 4.0 to 9.0 mM using an insulin (i.v. actrapid) sliding scale. The management of the patients was not affected by involvement in the study.

### Outcome Measure

Two clinical endpoints were collected: (i) survival on discharge from hospital and (ii) the Glasgow Outcome Score (GOS) at 3 months and 6 months after injury. The GOS was dichotomised into unfavourable recovery (GOS: 1 indicates death; 2, persistent vegetative state; and 3, severe disability) or favourable recovery (GOS: 4 indicates moderate disability; 5, good recovery) (Teasdale et al. (1998) J Neurotrauma 15:587-97).

### Sample Analysis

In each patient the microdialysis probe was allowed to stabilise for one hour (Bellander et al. (2004) supra). Only samples collected after this equilibration period were investigated. At the end of each one hour collection period the microdialysate of the cortical extracellular fluid was immediately analysed for lactate and pyruvate with the bedside CMA 600 Analyser (CMA Microdialysis) using an enzymatic colorimetric technique (Afinowi et al. (2009) Neuroscience Methods 181:95-9). For quality control analysis, artificial CSF samples were analysed on a calibrated YSI 2003 STAT Plus Glucose and Lactate Analyzer (YSI Inc., Yellow Springs, Ohio, USA) and compared to the values derived by the CMA 600 Analyser. The batch frozen samples were pooled in three hourly intervals and cortical extracellular fluid NfH levels were measured using an in-house developed and externally validated enzyme-linked immunosorbent assay (Petzold et al. (2003) J Immunol Methods 278:179-90; Petzold and Shaw (2007) J Immunol Methods 2007; 319:34-40). All samples were analysed in duplicates and repeated if the coefficient of variation (CV) was greater then the intra-assay precision of 10% (Petzold et al. (2003) supra). The analyst was blinded to all other information.

### Recovery

Recovery experiments were carried out as previously described (Afinowi et al. (2009) supra). Large volume (100 mL) CSF samples (n=12) were used as source solutions. Microdialysis pumps (CMA 106; CMA Microdialysis AB, Solna, Sweden) were used at a constant flow rate of 0.3 μL/min. The in vitro Relative Recovery (RR) was calculated as: Relative recovery (RR) (%)=dialysate concentration:reference solution concentration×100.

### Gel Electrophoresis

Following the NfH enzyme-linked immunosorbent assay, surplus of the cortical extracellular fluid samples was kept at 4° C. cortical extracellular fluid samples with high NfH content were pooled. Due to the small remaining sample size after performing the ELISA a total of 84 microvials from 5 patients were pooled to give 250 μL of cortical extracellular fluid fluid, know to have a high NfH concentration. The pooled cortical extracellular fluid was mixed with 170 μL Lithium Dodecyl Sulfate (LDS), 10 μL DTT and heated at 65° C. to unfold the proteins. The sample (400 μL) was loaded onto the 4-12% Bis-Tris and 3-8% Tris-Acetat gels. Molecular weight markers (Invitrogen, MagicMark, Western Standard and SeeBlue Plus2, Invitrogen) were used for each run. The gels were run under reducing conditions (Invitrogen System, PowerEase 500, 200 V, 120 mA, 25 W).

### Immunoblot

The proteins were transferred from the gel to a nitrocellulose membrane over a 2 hour blotting period (25 V, 160 mA, 17 W). The membrane was blocked in 2% fat milk powder/0.9% saline for 1 hour. Blocking solution was decanted and the membrane was washed 5 times for 3 minutes with washing solution (0.1% fat milk powder/0.9% saline, 0.1% tween 20). The membranes were either incubated with the first antibody in a single well or adjusted between two plastic layers and sealed in a manifold (hoefer) so that each of the 10 channels overlaid the entire area onto which the proteins have been transferred. The channel were filled with 3 mL of 0.1% fat milk powder/0.9% saline each, containing antibodies different antibodies in a 1:1000 dilution. For detection of NfH six mouse monoclonal antibodies directed at different phosphoepitopes (see Table 1 in Petzold et al. (2003) supra) were used: SMI 32, SMI 34, SMI 35, SMI 37, SMI 38 and SMI 311 (originally purchased from Sternberger Monoclonals Incorporated, now Covance Research Products, Berkeley Calif., USA). In addition antibodies to the neurofilament light chain (NfL) and medium chain (NfM) were also used (NR4 mouse monoclonal anti-NfL from Sigma and rabbit polyclonal anti-NfM from Affinity, UK). Antibodies were incubated at 4° C. overnight on a shaker. After decanting the antibodies, each channel was washed 5 times for 3 minutes with washing solution. The channels were then filled with the appropriate HRP-labelled detector antibody (swine anti-rabbit or rabbit anti-mouse, both Sigma) and incubated for one hour at room temperature. Antibodies were decanted and the membranes were washed in each channel for 5 minutes, 10 times. The membranes were incubated with the chemiluminescence substrate (SuperSingal West Pico, Thermo Scientific, #34078) for 5 minutes. The dried membranes were visualised on a AlphaEase Fluor Chem SP CCD camera.

### Dephosphorylation and Proteolysis

HPLC purified bovine NfH was diluted into Glycine buffer (pH 10.4) at a concentration of 50 μg/mL. NfH was dephosphorylated with 100 mU of alkaline phosphatase (ALP, EC 3.1.3.1, Sigma, P4252) at 37° C. for 1 hour (Petzold et al. (2008) Exp Neurol 213:326-35)). Aliquots of phosphorylated NfH and dephosphorylated NfH were incubated with (1) enterokinase (EK, EC 3.4.21.9, Roche, Cat No 11 334115001); (2) α-chymotrypsin (CT, EC 3.4.21.1, Sigma, C-4129).

### Laser Capture Microdissection

PC6-3 cells, a derivate from the PC12 cell line, were converted into neuronal-like cells as described by Pittman et al. (1993, J Neurosci 1993; 13:3669-80). The cells were grown in RPMI medium, 10% horse serum, 5% FCS and pen/strep. For differentiation the cells were switched to RPMI, 2% horse serum, 1% FCS+100 ng/ml NGF. The cells were then mounted on plolyethylene naphthalate membrane-covered glass slides. The cells were washed 3 times with PBS and fixed in 70% ethanol. The cells were air dried and the Laser Microbeam System (P.A.L.M., Microlaser Technologies AG, Munich, Germany) was used under visual control to capture the cells in barbitone buffer (pH 8.9) used for enzyme-linked immunosorbent assay (Schutze et al. (2007) Methods Cell Biol 82:649-73).

Protein Extraction from Snap Frozen Brain Tissue

Protein extraction was modified from (Ericsson et al. (2007) Acta Oncol, 46:10-20). LDS was added to the samples at 50 times the dry weight of the frozen brain tissue samples. All samples were sonicated on ice in a polypropylene tube. Samples were then incubated at 70° C. for 10 minutes. After rigorous mixing at 1400 rpm samples were centrifuged at 13.2×10³×g for 10 minutes at 4° C. The supernatant was used for western and immunoblotting.

### Immunohistochemistry

Sections (7 μm) were dewaxed and rehydrated followed by 15 min incubation in 3% hydrogen peroxide and deionised water followed by microwave heat treatment. Protein blocking was carried out with Normal Horse Serum. Sections were incubated overnight at room temperature with anti goat EK antibody (1:300 Santa Cruz Biotechnology, Inc., CA 95060, United States) using Vector ImPRESS™ anti goat detection system kit (Vector Labs: Burlington, Calif., USA) and visualised with diaminobenzidine (DAB) chromogen. Sections were washed with phosphate buffered saline (PBS)+0.05% Tween-20 in each step. Control sections were treated the same but the primary antibody was omitted. Neuronal apoptosis was assessed by staining for caspase-3 (1:500, Clone 269518, Code MAB 835, R&D System, Minneapolis, USA) as described (Sulejczak et al. (2008) Folia Neuropathol 46:213-9; Sairanen et al. (2009) Acta Neuropathol 118:541-52; Umschwief et al. (2010) J Cereb Blood FlowMetab 30:616-27).

### Immunofluoroescence

The same procedure was used as that for immunohistochemistry up until the incubation of the antibodies. Sections were incubated with EK antibody first at 1:400 dilution followed by secondary ImPRESS anti goat then Fluorescein Green Tyramide Signal Amplification (PerkinElmer Life and Analytical Sciences, Boston Mass.). After washing, the sections were quenched with 1% hydrogen peroxide for 15 min in order to prevent any deposited tyramide combining with the second tyramide signal that followed. After washing, protein blocking was done with normal horse serum (Vector laboratories) followed by incubation with primary monoclonal antibody SMI32 (1:500, Covance, Emeryville, Calif., USA) and monoclonal neurofilament 200 (NE14, 1:3000, Sigma, St Louis, Mo. 63103, USA). Sections were incubated with relevant secondary HRP detection system followed by CY3 from the Tyramide Signal Amplification Kit. Sections were mounted on Vectashield® with Dapi (Vector laboratories) and visualised with a Leica confocal laser microscope. After each step sections were washed with PBS. Negative controls were treated the same except the primary antibodies were omitted.

### Data Analysis

Statistical analyses were performed using SAS software (V9.2). Independent variables were compared using the non-parametric two-sample exact Wilcoxon rank-sum test for two variables and a two-way unbalanced ANOVA (general linear model) for more than two variables. Repeated measurement analysis was performed on 3 h epochs (pooled cortical extracellular fluid sampling time) using mixed models. The best fitting model was selected based on the best fitting covariance structure (Littell et al. (1998) J Anim Sci 76:1216-31). The linear relationship between continuous variables was evaluated using the Spearman correlation coefficient. The level of significance for the multiple correlations was corrected using the Bonferroni method. The Chi-Square-test and Fisher exact test were used for comparing proportion of patients. Two-tailed tests were used throughout and P-values of <0.05 were accepted as significant.

### Results

### Neurofilaments are a Protein Biomarker for Neuronal Loss

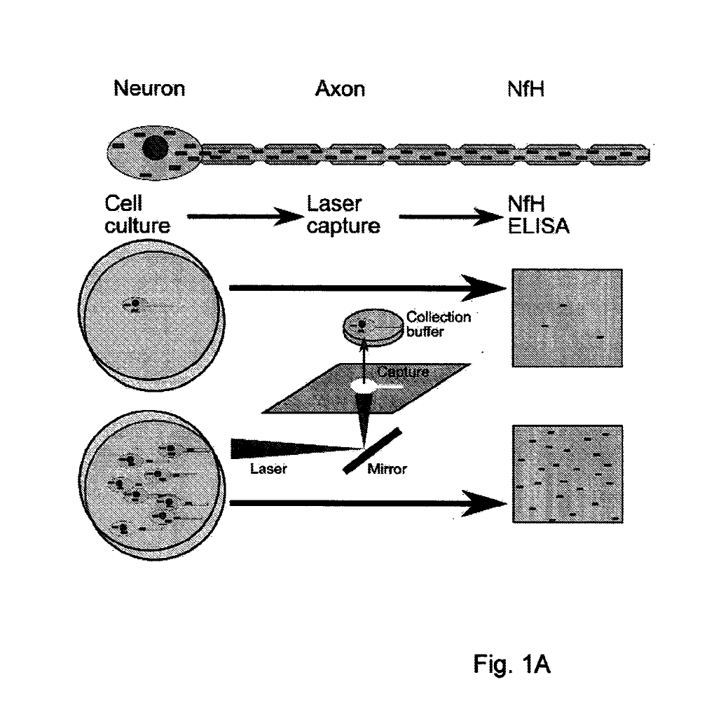

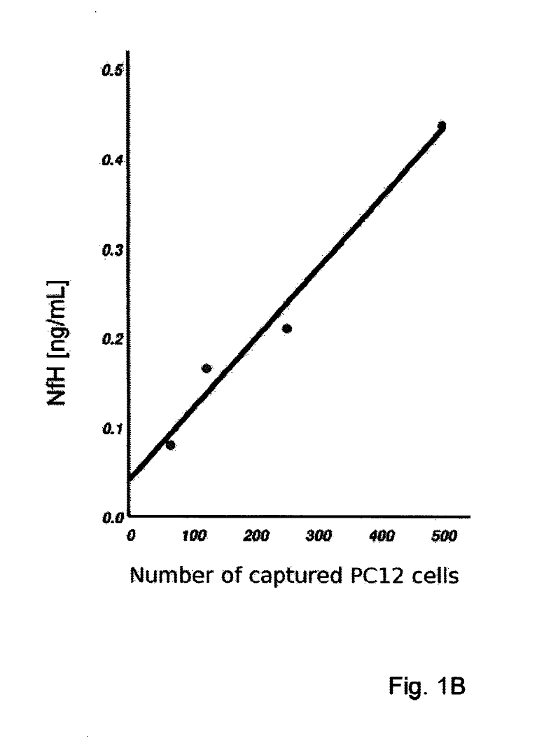

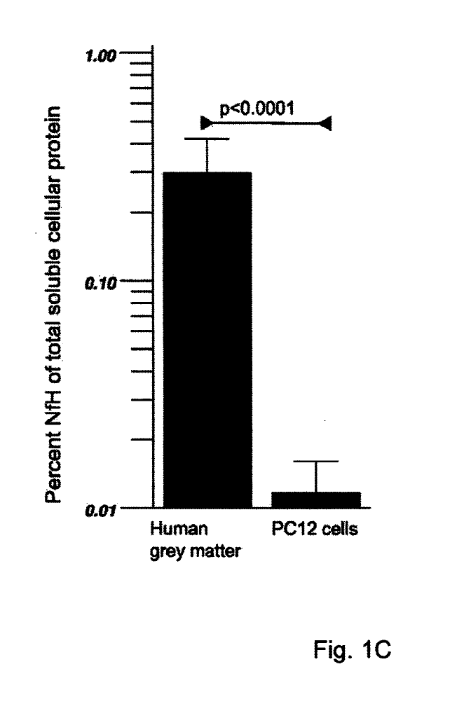

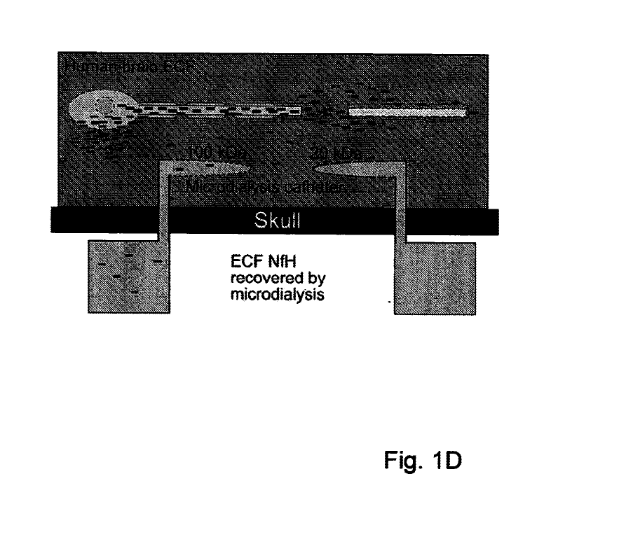

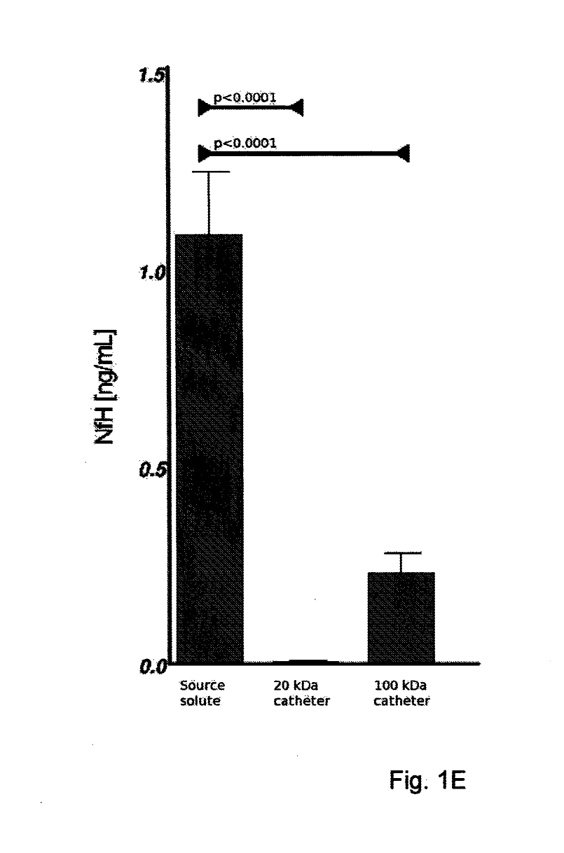

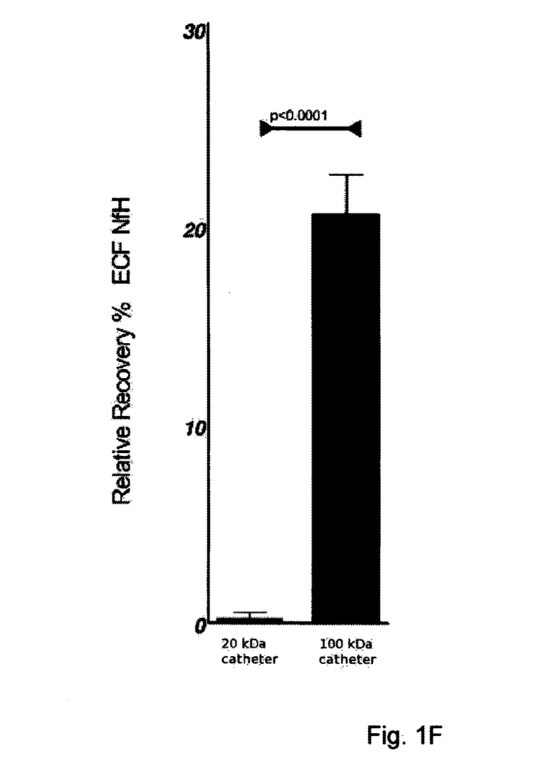

As a proof of principle, an in vitro cell culture setup was used to investigate the relationship between the NfH protein concentration and the number of PC12 cells (See FIG. 1A). Individual PC12 cells were collected using laser-capture microdissection (Schutze et al. (2007) Methods Cell Biol 2007; 82:649-73). A minimum of 50 PC12 cells were needed for detection of NfH in the enzyme-linked immunosorbent assay (Petzold et al. (2003) J Immunol Methods 278:179-90; Petzold and Shaw (2007) J Immunol Methods 2007; 319:34-40). The linear relationship (R=0.98, P=0.013) of laser-captured PC12 cells with NfH levels equated to: PC12 cells number=1245×NfH−46 (FIG. 1B). In PC12 cells, NfH accounted only to 0.011% of the total soluble cellular protein fraction which was 24.6 times less than the 0.271% quantified from human grey matter (P=0.0014) (FIG. 1C).

The relative recovery of NfH from a solute is influenced by the pore size of the microdialysis membrane (FIG. 1E). The in vitro recovery experiment showed a significant loss of NfH from the source solute compared with samples recovered either through a 20 kDa or 100 kDa microdialysis membrane (general linear model, F(2,33)=171, P<0.0001, FIG. 1F). The mean relative recovery of NfH for the 100 kDa membrane (CMA71, 20 mm length, 0.6 mm diameter polyamide membrane) was 20.7±3.2 (FIG. 1F). For the 20 kDa membrane (CMA70, 20 mm length, 0.6 mm diameter polyamide membrane) the relative recovery was negligible at 0.2±0.4% (Kruskal-Wallis, X²=18.23, P<0.0001).

NfH Cleavage Products are Recovered from Human ECF

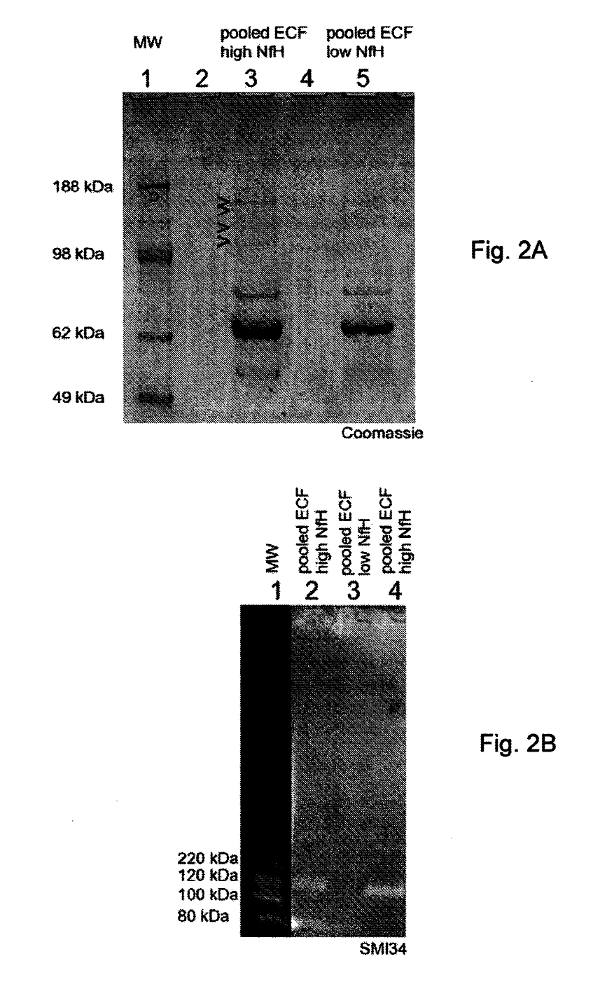

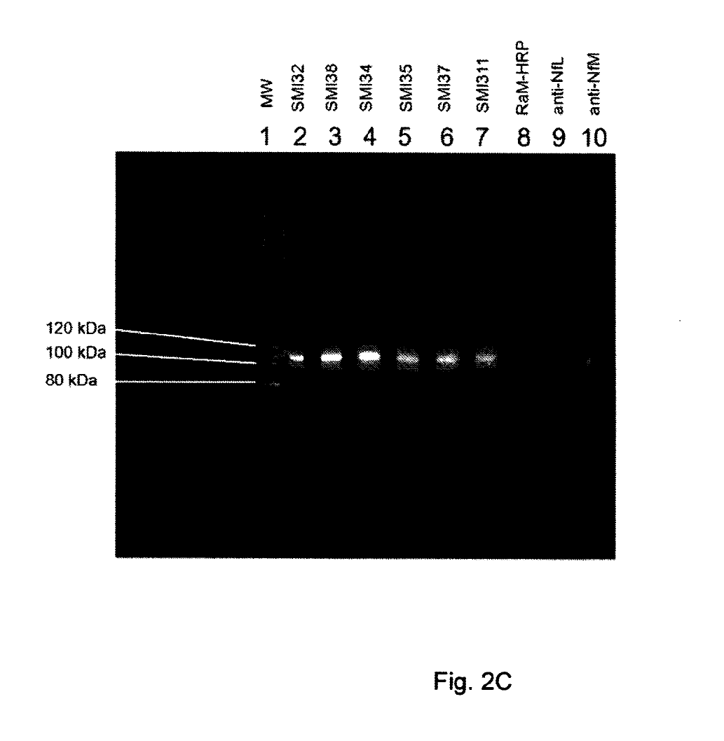

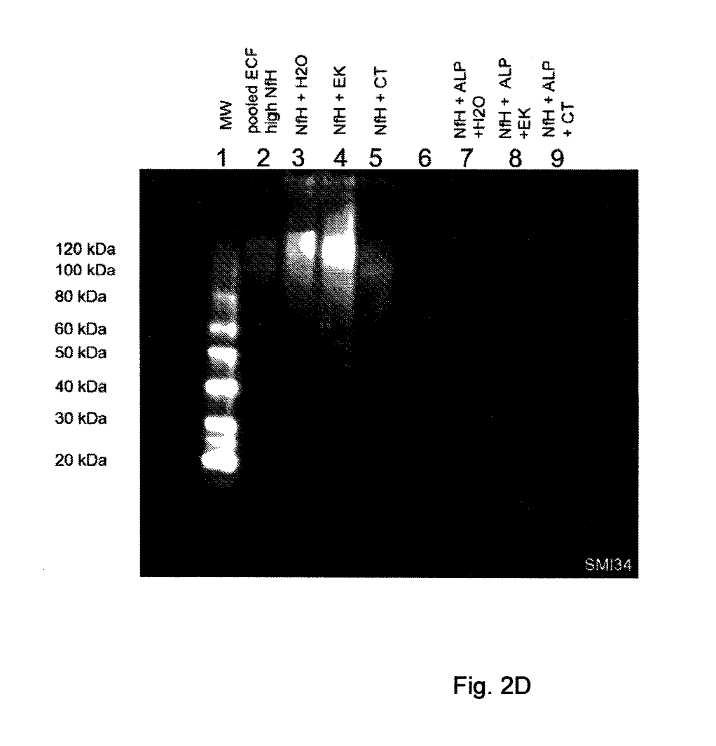

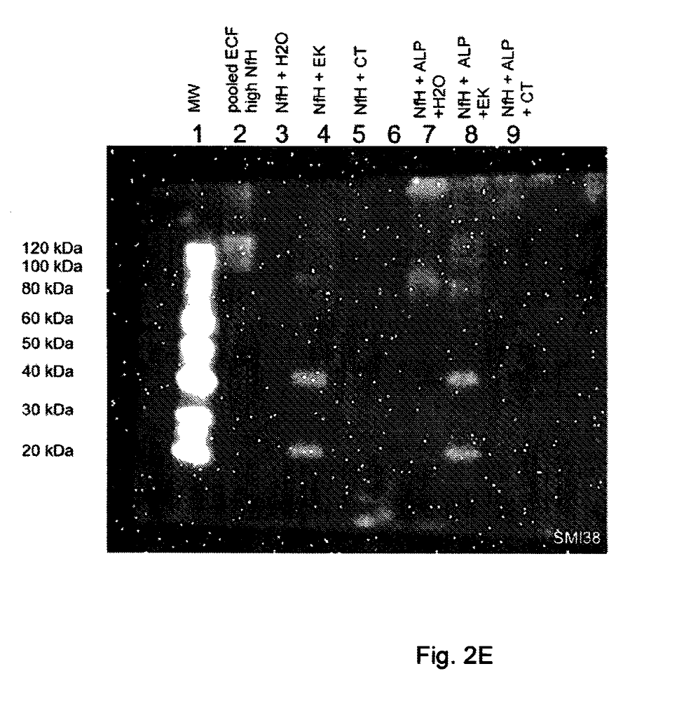

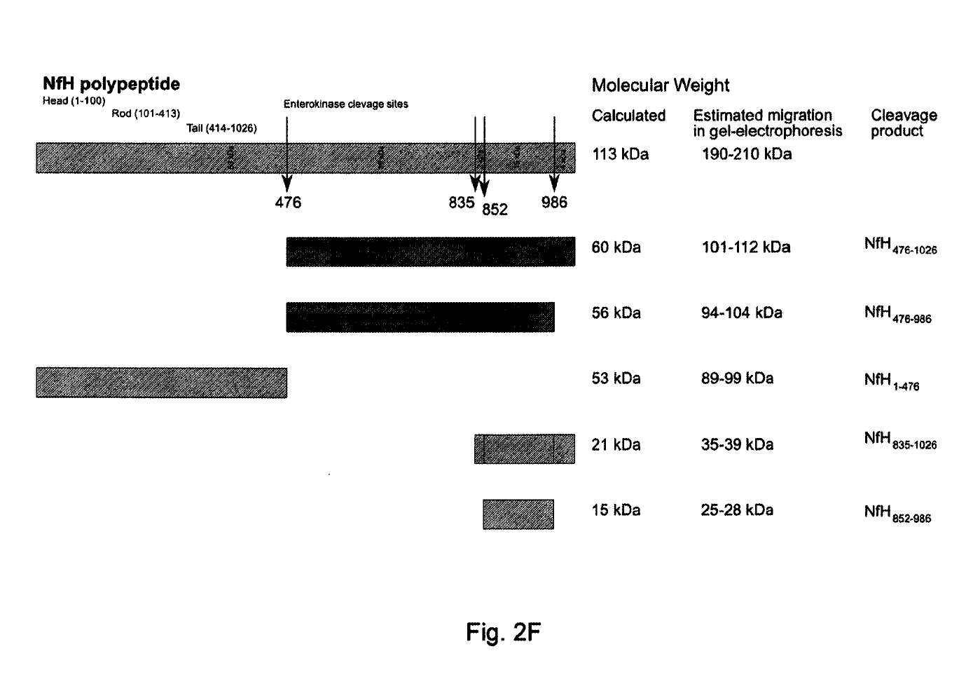

Pooled human extracellular fluid (ECF) with high and low NfH levels were used to investigate how this large protein (1020 amino acids corresponding to a molecular mass of 113 kDa) could be recovered through a 100 kDa catheter membrane. Of note, most of the literature refers to the molecular mass derived from sodium dodecyl sulphate (SDS) gels which is in the 190-210 kDa range. The difference between these molecular weight values is explained by altered electrophoretic mobility caused by the charge/weight of the bound phosphate. Using gelelectrophoresis it was demonstrated that high molecular weight proteins were present in extracellular fluid with high NfH levels but not in ECF with low NfH levels (FIG. 2A). The immunoblot showed immunoreactivity of a protein with SMI34 (FIG. 2B). The immunoreactivity appears to be specific for the NfH phosphoforms SMI32, SMI38, SMI34, SMI35, SMI37 and SMI311, as there was no cross-reactivity with antibodies against NfM and NfL (FIG. 2C). Comparative immunoblotting identifies two NfH cleavage products in the 100-120 kDa range (FIG. 2D, lane 2 and FIG. 2E, lanes 2 and 7).

The described in vivo cleavage products can be reproduced in vitro by incubation of purified NfH with enterokinase (FIG. 2D, lane 4 and FIG. 2E, lane 8). The electrophoretic mobility of the extracellular fluid NfH cleavage products (NfH₄₇₆₋₉₈₆ and NfH₄₇₆₋₁₀₂₆) matches what can be expected from the known NfH EK cleavage sites (FIG. 2F). The corresponding molecular weight of NfH₄₇₆₋₉₈₆ and NfH₄₇₆₋₁₀₂₆ calculated from the amino acid sequence of the cleavage product corresponds to 56-60 kDa (FIG. 2F). Herein, the extracellular fluid NfH cleavage products will be referred to as eNfH (for extracellular fluid NfH).

### A Novel Neuronal Proteolytic Pathway

The immunohistochemistry shows the presence of EK in the frontal cortex of a patient with traumatic brain injury (FIG. 3A). Mainly large neurons and their axons are stained. There was almost no immunoreactivity to enterokinase in the deep white matter (data not shown). EK was not found in endothelial cells, astrocytes or microglia. The exclusively neuronal expression of EK is not specific for TBI because a similar staining pattern is also seen in patients with epilepsy (FIG. 3B). The immunuhistochemistry suggests that the heavy subunit of enterokinase may be membrane bound. This observation is confirmed by the immunofluoroescence (FIG. 3C and 3D). The predominant expression of the enterokinase heavy chain in the human cortex, but not deep white matter, is confirmed by immunoblotting (FIG. 3E). In a subpopulation of large neurons EK colocalises with phosphorylated NfH (FIG. 3C).

### Apoptosis of Cortical Neurons in TBI

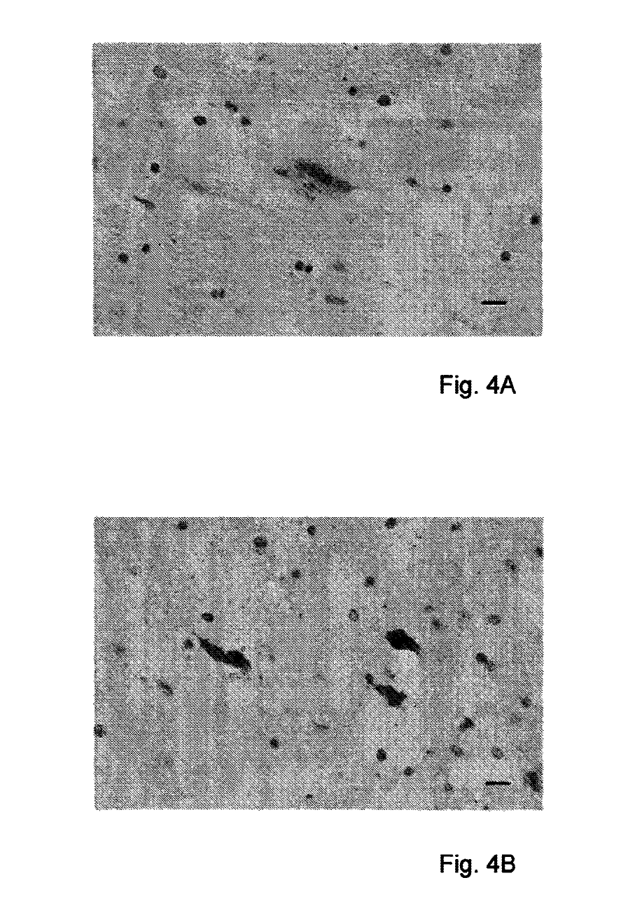

In all traumatic brain injury post-mortem cases there are apoptotic caspase-3 positive cells in the vicinity of the contusion (Table 1). Staining for caspase-3 was essentially absent from normal control PM tissue and temporal lobar tissue removed during epilepsy surgery (Table 1). The morphological analysis of the Caspase-3 cells identifies different types of neurons including pyramidal cells (FIGS. 4A and B). Many of the Caspase-3 positive cells were degenerate and apoptotic.

TABLE 1
Neuronal apoptosis in traumatic brain injury

| ID | Age (gender) | Tissue | Brain area | Condition | Caspase-3 |
|---|---|---|---|---|---|
| Patient 1 | 85 (F) | Post-mortem | Contusion, SDH, ICH | Traumatic brain injury | + |
| Patient 2 | 73 (F) | Post-mortem | Contusion | Traumatic brain injury | + |
| Patient 3 | 83 (M) | Post-mortem | Contusion | Traumatic brain injury | + |
| Patient 4 | 51 (M) | Post-mortem | Contusion | Traumatic brain injury | + |
| Patient 5 | 39 (F) | Post-mortem | Temporal lobe | Normal control | − |
| Patient 6 | 35 (F) | Post-mortem | Temporal lobe | Normal control | ± |
| Patient 7 | 48 (M) | Surgery | Temporal lobe | Epilepsy | − |
| Patient 8 | 34 (M) | Surgery | Temporal lobe | Epilepsy | − |
| Patient 9 | 47 (F) | Surgery | Temporal lobe | Epilepsy | − |

Neuronal staining for caspase-3:
− = absent,
± = uncertain,
+ = positive.
ICH = intra-cranial haemorrhage;
SDH = subdural haematoma.

### Patients

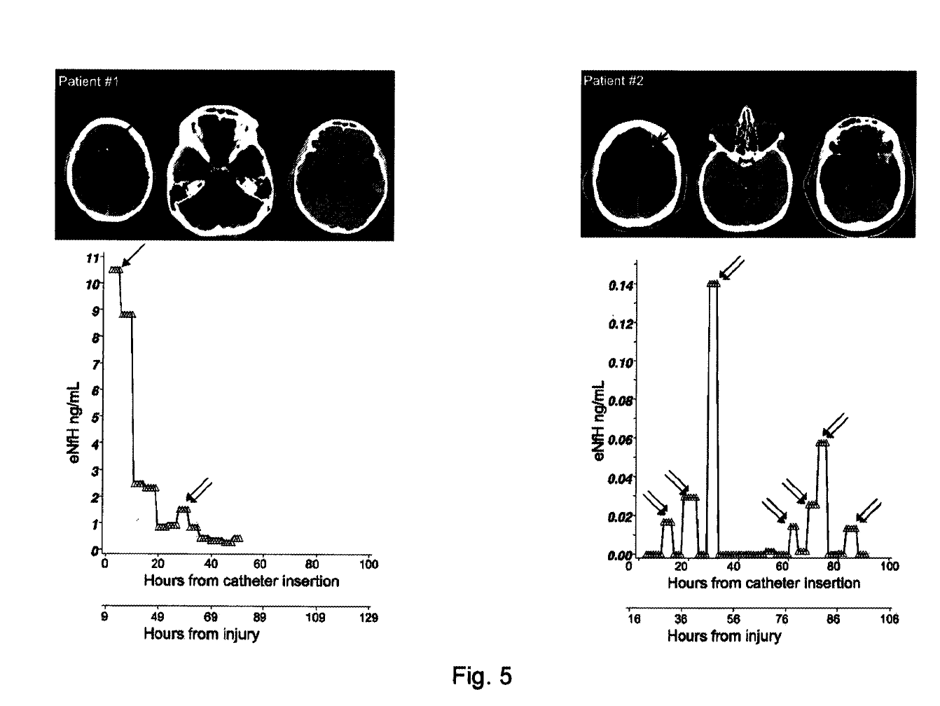

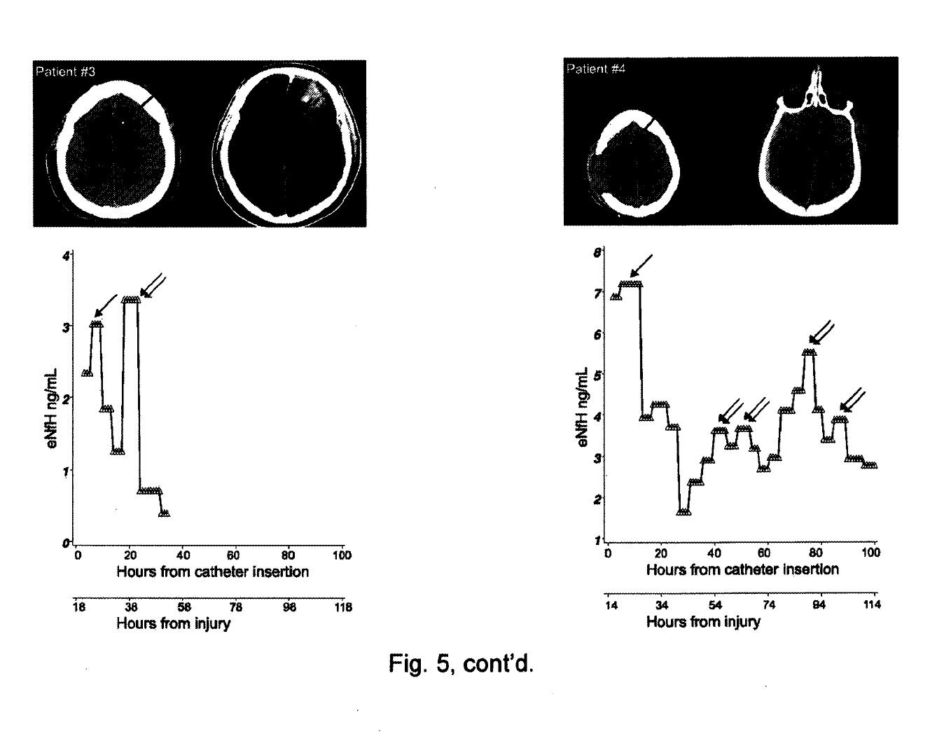

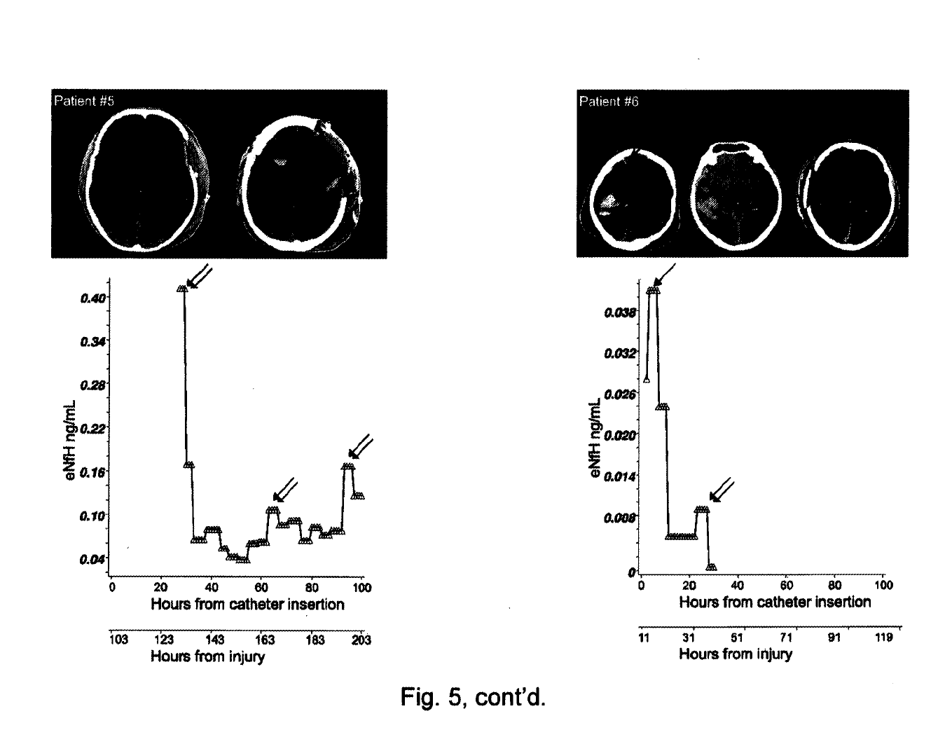

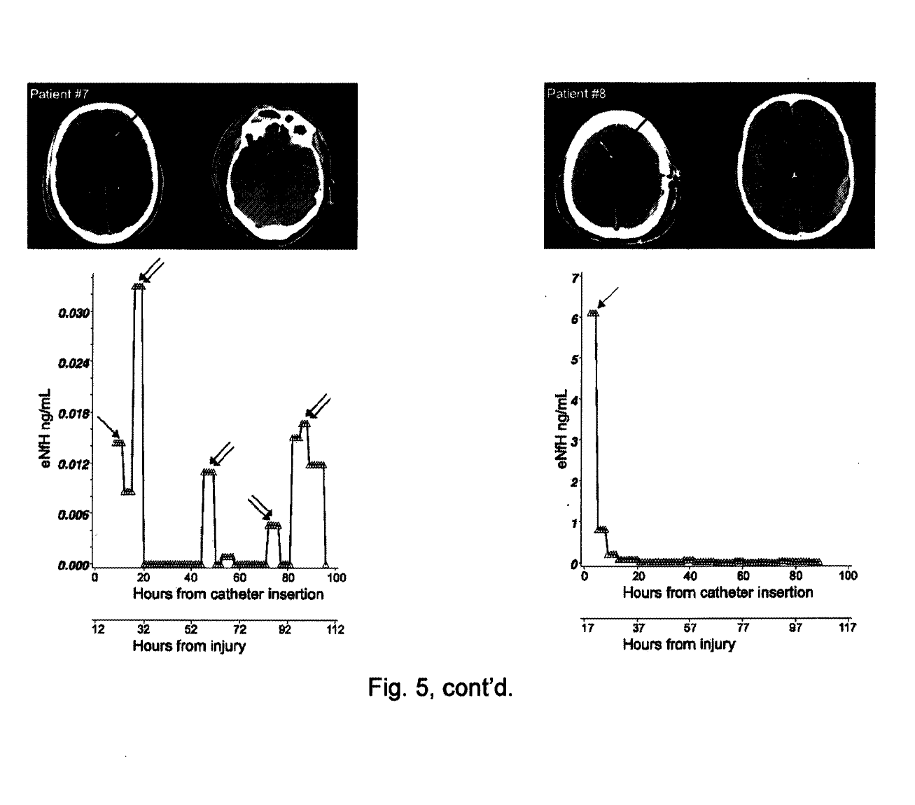

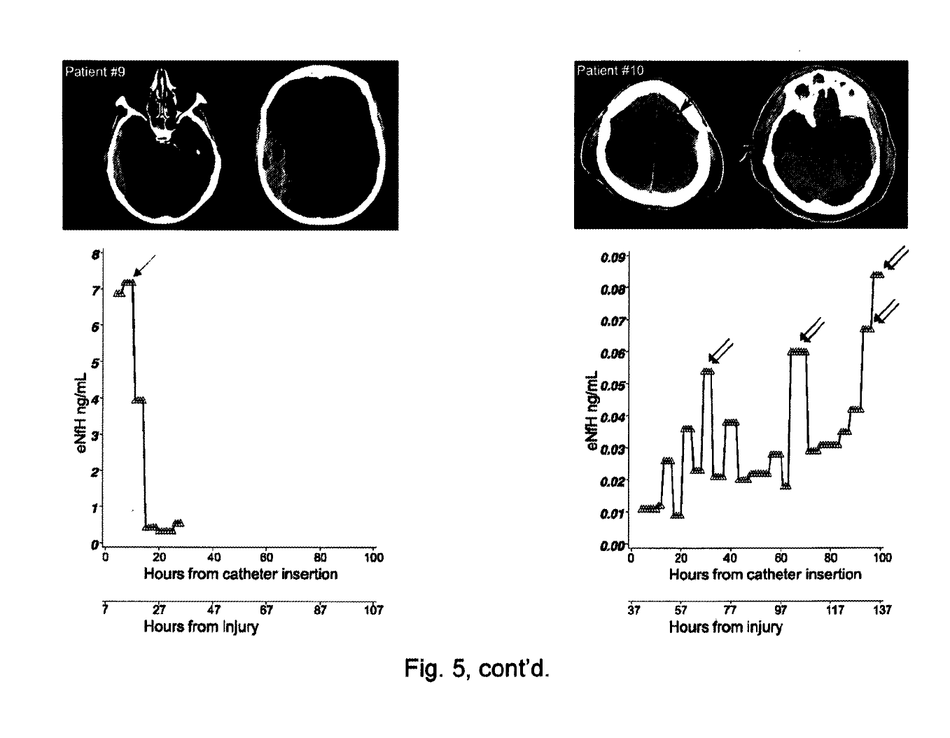

The clinical characteristics of the 10 patients with traumatic brain injury (TBI) recruited into this prospective, longitudinal study were scored. Nine patients were male and one was female. The median age was 39 years (range 20-68 years). The severity of the injury ranged from mild to severe, with a median admission GCS of 9 (range 4-15). The individual CT brain scans documenting the brain pathology and the position of the microdialysis catheter are shown in FIG. 5.

### Extracellular Fluid Neurofilament Heavy Chain in Traumatic Brain Injury

A total of 640 microdialysis samples were collected on an hourly basis for this study. The median time between injury and catheter insertion was 14 hours (range 7 to 37 hours) when considering all but one patient (#5) who was referred to us with a 5 day delay.

The individual eNfH levels are shown in FIG. 5. An early peak of the eNfH concentration was observed in 7/10 (70%) of patients (#1, #3, #4, #6, #7, #8, #9; indicated by single arrows in FIG. 5). In addition, secondary peaks of eNfH were observed in 8 patients (#1, #2, #3, #4, #5, #6, #7, #10; indicated by double arrows in FIG. 5). In 4 patients the concentration of eNfH measured in secondary peaks during the later disease course exceeded what was observed initially (#2, #3, #7, #10). Due to small sample volume in patient #5 it was only possible to measure eNfh 25 hours after catheter insertion. This patient shows an early peak of eNfH at around 0.4 ng/mL. The longitudinal data on ECF lactate and ECF pyruvate values in this patient, directly following catheter insertion suggests that this eNfH peak was not likely a consequence of a catheter insertion artifact.

### Phenotypic Analyses

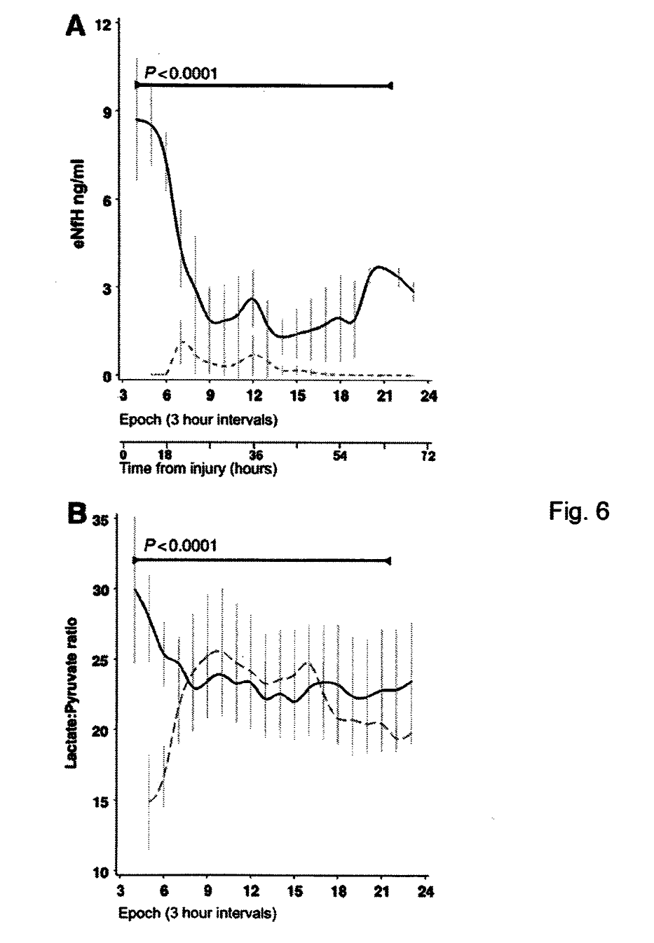

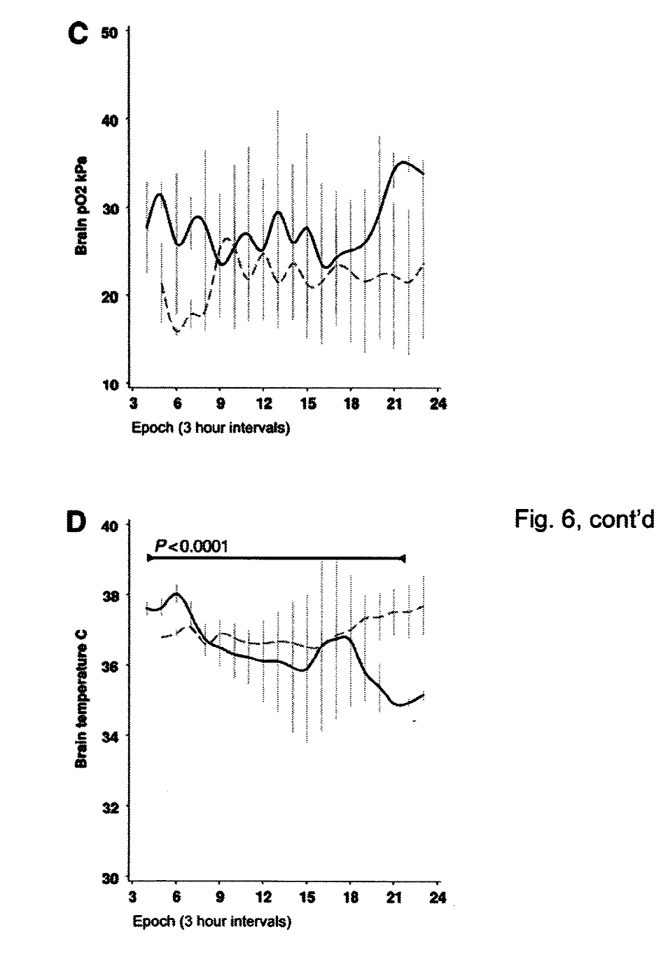

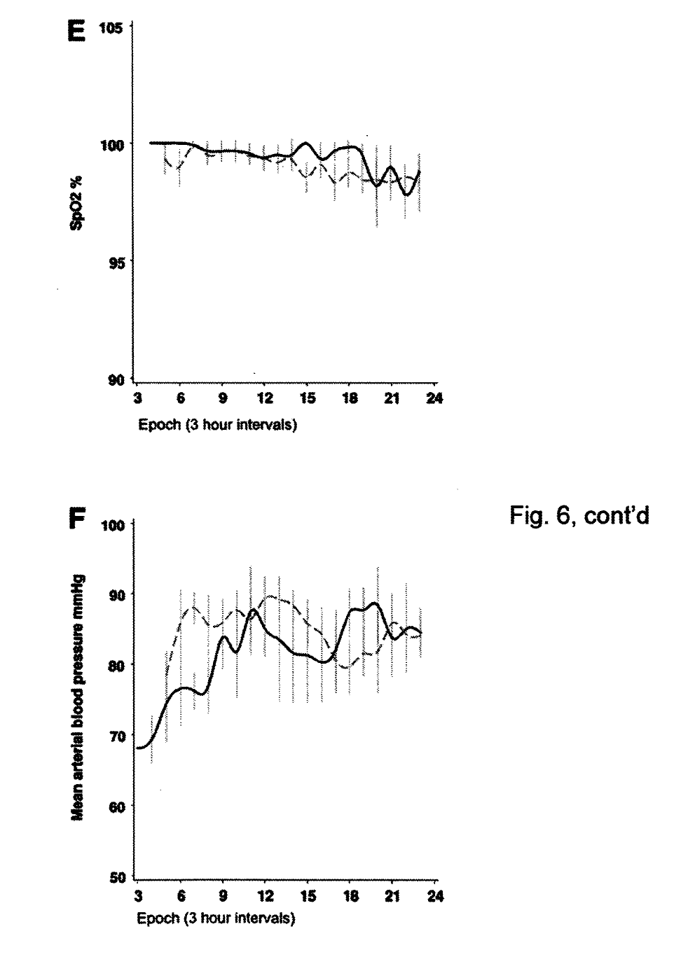

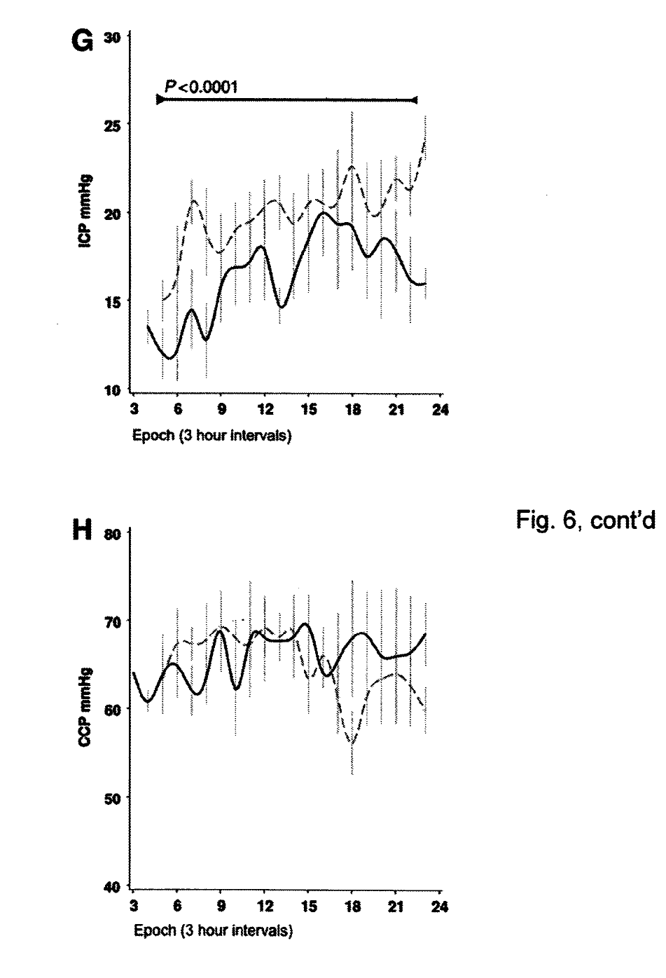

Road traffic accidents were classified in three patients and assault in one patient (Patient 5) as high-velocity impact traumatic brain injury. However, the actual impact of injury was not recorded (see below). A fall was the cause of traumatic brain injury in the remaining six patients. Patients who suffered from high-velocity impact traumatic brain injury had considerably higher early extracellular fluid NfH levels compared with those suffering from a fall. The averaged extracellular fluid NfH levels over the initial 24 h were 7-fold higher (6.18±2.94 ng/ml) following high-velocity impact traumatic brain injury compared with a fall (0.84±1.77 ng/ml). Moreover, extracellular fluid NfH levels remained for longer on a high level following high-velocity impact traumatic brain injury [F(19,344)=28.72, P<0.0001, FIG. 6A]. Following a fall, the extracellular fluid NfH levels declined steadily to become non-detectable after an average of 2 days.

In patients with a high-velocity impact traumatic brain injury, the extracellular fluid lactate to pyruvate ratio was above 25 at onset and the longitudinal profile was different compared with the lactate to pyruvate ratio in traumatic brain injury after fall [F(18, 366)=3.67, P<0.0001, FIG. 6B]. Similarly, there was a mild but significant difference in the brain temperature profile [F(18, 297)=5.89, P<0.0001, FIG. 6D]. Finally, the intra-cranial pressure remained higher in patients with traumatic brain injury with a fall compared with high-velocity impact traumatic brain injury throughout the entire observation period [F(18, 370)=1.94, P=0.012, FIG. 6G]. None of the other physiological parameters distinguished between the two groups (FIGS. 6C, E, F and H).

Because the impact of injury was not recorded, additional analyses were performed comparing three categories: road traffic accidents, assault and falls. There was a significant overall difference between these three groups over time for extracellular fluid NfH levels (F=3.41, P=0.0049) and brain temperature (F=8.13, P<0.0001). However, significance was lost for the extracellular fluid lactate to pyruvate ratio (F=0.35, P=0.91) and intra-cranial pressure (F=0.50, P=0.81).

Injury severity was classified according to the GCS into mild (n=3), moderate (n=2) and severe (n=5) traumatic brain injury. There was no significant difference for extracellular fluid NfH levels according to the GCS [F(5, 481)=0.14, P=0.98].

### Physiological Correlations

A correlation analysis was performed using the multimodal monitoring data set focusing on four physiological parameters which have been are related to injury severity and outcome in traumatic brain injury (Enblad et al., 2001 Stroke 32:1574-80); Hutchinson et al., 2002 J Cereb Blood Flow Metab 22:735-45; Butcher et al., 2007 Neurotrauma 24:294-302; Arieli et al., 2008 Eur J Appl Physiol 104:867-71; Belli et al., 2008 Acta Neurochir 150:461-9; Diringer, 2008 Curr Opin Crit. Care 14:167-71; Polderman, 2008 Lancet 371:1955-69; Stiver and Manley, 2008 Neurosurg Focus 25:E5). FIG. 7A shows the statistical strength of the correlation (Spearman's R) between these parameters and eNfH levels.

### Lactate to Pyruvate Ratio

There was a strong and significant correlation between the lactate to pyruvate ratio and extracellular fluid NfH levels in the initial three epochs (R=0.83 to R=0.63, FIG. 7A). The raw data for the second epoch show that extracellular fluid NfH levels started to rise with a lactate to pyruvate ratio>20 (FIG. 7B).

### Systemic Blood Pressure

The mean arterial blood pressure correlated significantly with extracellular fluid NfH levels between the second and fifth epoch (R=−0.75 to R=−0.46, FIG. 7A) and during the later course when cerebral perfusion pressure management became critical (FIG. 6H). During the epoch where the correlation of the mean arterial blood pressure with extracellular fluid NfH levels was the strongest (Epoch 4, R=−0.75, P<0.0001), there was also an inverse correlation of the cerebral perfusion pressure with extracellular fluid NfH levels (R=−0.67, P=0.0001, FIG. 7A). The raw data show the highest extracellular fluid NfH levels for a mean arterial blood pressure below 90 mmHg (FIG. 7D).

### Brain Oxygenation

There was a moderate correlation of the brain tissue oxygenation with extracellular fluid NfH levels (R=0.85 to R=0.73, FIG. 7A). The raw data for the second epoch show that this correlation was due to a scatter of high CSF NfH levels for brain tissue oxygenation levels above 30 kPa (FIG. 7F).

### Brain Temperature

There was a significant correlation between higher brain temperature and extracellular fluid NfH levels (R=0.72 to R=0.51, FIG. 7A). The raw data show the highest extracellular fluid NfH levels for a brain temperature above 37° C. (FIG. 7H).

### Prognosis

Five (50%) of the 10 patients with traumatic brain injury died within a median of 5 days (range 4-6 days) following the injury.

The individual percentage of hours with pathological values for the sampled physiological parameters spanning the entire observation period (up to 230 hours post TBI) were scored. These physiological parameters were more frequently pathological in non-survivors compared to survivors for ICP (p<0.0001), lactate to pyruvate ratio>25 (p=0.0003) or lactate to pyruvate ratio>40 (p<0.0001), brain tissue oxygenation (BtO2) (p<0.0001) and brain temperature (BT) (p<0.0001), but not for the CPP (p=0.84). Non-survivors had significantly more periods where the mean hourly lactate to pyruvate ratio was above our cut-off of 25 or Vespa's more conservative cut-off of 40.

The initial prognostic value of these physiological parameters and of eNfH levels were investigated with logistic regression models using survival as the outcome variable. Models were tested for validity based on the minimum observation period needed to reveal any meaningful prognostic information. After a 12 hour observation period the best predictor for mortality were eNfH levels with an odds ratio of 7.68 (CI 2.15-27.46). The other two strong predictors of mortality were the ICP (5.06, CI 1.84-13.89) and admission GCS (4.13, CI 1.72-9.94).

1. A biomarker for the detection of brain damage or a disease associated with loss of neurons, said biomarker comprising a protein fragment of the neurofilament heavy chain (NfH) protein in a biological sample, wherein said protein fragment is a polypeptide selected from the group consisting of:
i) a polypeptide consisting of the amino acid sequence selected from the group consisting of SEQ ID NO:1, SEQ ID NO:2, SEQ ID NO:3, SEQ ID NO:4 and SEQ ID NO:5, more preferably of SEQ ID NO:2, SEQ ID NO:3, SEQ ID NO:4 and SEQ ID NO:5, most preferably the polypeptides composed of amino acids 476-1026 and 476-986 of SEQ ID NO 6;
ii) a protein fragment of the NfH protein consisting of the amino acid sequence that is at least 60% identical to SEQ ID NO:1, at least 60% identical to SEQ ID NO:2, at least 60% identical to SEQ ID NO:3, at least 60% identical to SEQ ID NO:4 or at least 60% identical to SEQ ID NO:5;
iii) an Enterokinase cleavage product of the NfH protein; and
iv) a polypeptide that is a encoded by an allelic variant of the nucleic acid coding for a protein having the amino acid sequence selected from the group consisting of SEQ ID NO:1, SEQ ID NO:2, SEQ ID NO:3, SEQ ID NO:4 and SEQ ID NO:5, more preferably of SEQ ID NO:2, SEQ ID NO:3, SEQ ID NO:4 and SEQ ID NO:5, most preferably the polypeptides composed of amino acids 476-1026 and 476-986 of SEQ ID NO 6.

2. Biomarker according to claim 1, wherein said protein fragment is an Enterokinase cleavage product having a molecular weight selected from the group consisting of 101-112 kDa or 94-104 kDa, 89-99 kDa, 35-39 kDa and 25-28 kDa, more preferably of 101-112 kDa and 94-104 kDa, as determined by migration distance in gel-electrophoresis.

3. Biomarker according to claim 1, wherein said Enterokinase cleavage product of the NfH protein is a polypeptide selected from the group consisting of a polypeptide with a calculated molecular weight of about 54 kDa having the amino acid sequence of SEQ ID NO:1, a polypeptide with a calculated molecular weight of about 39 kDa having the amino acid sequence of SEQ ID NO:2, a polypeptide with a calculated molecular weight of about 2 kDa having the amino acid sequence of SEQ ID NO:3, a polypeptide with a calculated molecular weight of about 15 kDa having the amino acid sequence of SEQ ID NO:4, a polypeptide with a calculated molecular weight of about 4 kDa having the amino acid sequence of SEQ ID NO:5, a polypeptide with a calculated molecular weight of about 60 kDa having the amino acid sequence of SEQ ID NO:14, a polypeptide with a calculated molecular weight of about 56 kDa composed of the amino acid sequence of SEQ ID NO 15, a polypeptide with a calculated molecular weight of about 41 kDa composed of amino acids 476-852 of SEQ ID NO 6, a polypeptide of about 21 kDa composed of amino acids 835-1026 of SEQ ID NO 6, a polypeptide of about 17 kDa composed of amino acids 835-986 of SEQ ID NO 6, a polypeptide of about 19 kDa composed of amino acids 852-1026 of SEQ ID NO 6, a polypeptide of about 92 kDa composed of amino acids 1-835 of SEQ ID NO 6, a polypeptide of about 94 kDa composed of amino acids 1-852 of SEQ ID NO 6, a polypeptide of about 109 kDa composed of amino acids 1-986 of SEQ ID NO 6, a polypeptide that is at least 60% identical to the indicated polypeptides and/or that are encoded by a naturally occurring allelic variant of the nucleic acid coding for NfH.

4. A method for determining the level of a biomarker for brain damage or a disease associated with loss of neurons in a subject, said method comprising steps of:
a) measuring in a biological sample of said subject or a purified fraction thereof the amount of a biomarker according to claim 1;
b) comparing the amount of said biomarker in said biological sample with a reference value, wherein an elevated level of said biomarker is indicative of the presence or the progression of said brain damage or a disease associated with a loss of neurons.

5. Method according to claim 4, wherein said reference values are values for healthy subjects in the case of determining the presence of said brain damage or a disease associated with a loss of neurons, or values of the same subject at earlier points in time in the case of prognosis of said brain damage or a disease associated with a loss of neurons.

6. Method according to claim 4, wherein said biological sample is a sample selected from the group consisting of cortical brain tissue, plasma, serum, amniotic fluid, urine, vitreous body, cerebrospinal fluid and blood.

7. Method according to claim 6, wherein said biological sample is a microdialysate of the extracellular fluid of the brain, preferably obtained using a microdialysis membrane having a cut-off of 15-20 kDa, preferably 15-100, more preferably around 100 kDa.

8. Method according to claim 4, wherein measuring the amount of said biomarker is performed by a method selected from the group consisting of:
a) adding to said biological sample or a purified fraction thereof an antibody or a capture agent that binds specifically to said biomarker to allow said biomarker to form a complex with said specific antibody and detecting said complex;
b) subjecting said biological sample or a purified fraction thereof to an ionizing treatment to allow said biomarker to generate charged molecules or molecule fragments, measuring the mass-to-charge ratio of said charged molecules or molecule fragments and identifying the biomarker based on the measured mass-to-charge ratio.

9. Method according to claim 4, wherein said disease is a neurodegenerative disease.

10. Method according to claim 4, wherein said brain damage is TBI, preferably high impact TBI.

11. Method according to claim 4, wherein said biological sample is collected within a week, preferably within 12 hours after the onset of said brain damage or a disease associated with a loss of neurons.

12. A method according to claim 4, whereby said biomarker is detected by an electrochemiluminescence (ECL)-based immunoassay.

13. Method for preparing a biomarker as defined in claim 1, comprising steps of:
a) combining a NfH protein and Enterokinase in an aqueous solution;
b) allowing the cleavage of said NfH protein; and
c) optionally isolating the resulting protein fragment

14. A biomarker as prepared according to the method of claim 13.

15. Use of a biomarker as defined in claim 1 as a reference protein in a test for detecting neuronal loss in a sample of a subject.

16. An antibody or a molecule having the antigen-binding portion of an antibody which specifically detects a biomarker according claim 1.

17. A kit of parts comprising a biomarker as defined in any one of claims 1-3 or 14 and/or an antibody according to claim 16, optionally in combination with a written instruction for performing a method according to claim 4.
